(19) United States
(12) Patent Application Publication
(10) Pub. No.: US 2025/0299812 A1
(43) Pub. Date: Sep. 25, 2025

(54) SYSTEMS AND METHODS FOR MEDICAL CARE THROUGH DATA MONITORING AND FEEDBACK TREATMENT

(71) Applicant: Etiometry Inc., Boston, MA (US)

(72) Inventors: Dimitar V. Baronov, Weston, MA (US); Evan J. Butler, New Haven, CT (US); Jesse M. Lock, Winchester, MA (US)

(21) Appl. No.: 19/084,392

(22) Filed: Mar. 19, 2025

Related U.S. Application Data
(60) Provisional application No. 63/567,096, filed on Mar. 19, 2024.

(51) Int. Cl. G16H 40/63; G16H 10/60; G16H 20/00; G16H 50/20

(52) U.S. Cl. CPC G16H 40/63; G16H 10/60; G16H 20/00; G16H 50/20

(57) ABSTRACT

Systems, methods, and computer-readable media for providing a decision support solution to medical professionals to determine medical care through data monitoring and feedback treatment are provided herein. In another embodiment, a computer-implemented method for modeling patient outcomes resulting from treatment in a specific medical area includes receiving patient-specific data associated with a patient, determining a plurality of possible patient states under which the patient can be categorized, a current patient state under which the patient can be categorized and determining probabilities of the patient transitioning from any of the possible patient states to every other possible patient state.

### RELATED APPLICATIONS

This application claims priority to U.S. provisional patent application 63/567,098, titled “Systems and methods for medical care through data monitoring and feedback treatment” filed Mar. 19, 2024 and naming Dimitar V. Baronov, Evan J. Butler, and Jesse M. Lock as inventors [Attorney Docket No. 3816-11804].

This application is related to U.S. patent application Ser. No. 18/083,949, titled “Systems and methods for optimizing medical care through data monitoring and feedback treatment,” filed Dec. 19, 2022 and naming Dimitar V. Baronov, Evan J. Butler, and Jesse M. Lock as inventors [Attorney Docket No. 3816-11803],
which is a continuation of U.S. patent application Ser. No. 15/881,255 and titled “Systems and methods for optimizing medical care through data monitoring and feedback treatment,” filed Jan. 26, 2018 and naming Dimitar V. Baronov, Evan J. Butler, and Jesse M. Lock as inventors, issued Jan. 17, 2023 as U.S. Pat. No. 11,557,294 [Attorney Docket No. 3816-11801],
which is a continuation of U.S. patent application Ser. No. 14/535,149 titled “Systems and methods for optimizing medical care through data monitoring and feedback treatment,” filed Nov. 6, 2014 and naming Dimitar V. Baronov, Evan J. Butler, and Jesse M. Lock as inventors [Attorney Docket No. 3816-11601],
which is a continuation of U.S. patent application Ser. No. 13/689,029 titled “Systems and methods for optimizing medical care through data monitoring and feedback treatment,” filed Nov. 29, 2012 and naming Dimitar V. Baronov, Evan J. Butler, and Jesse M. Lock as inventors [Attorney Docket No. 3816-10501],
which is a continuation of U.S. patent application Ser. No. 13/698,319 titled “Systems and methods for optimizing medical care through data monitoring and feedback treatment,” filed Nov. 16, 2012 and naming Dimitar V. Baronov, Evan J. Butler, and Jesse M. Lock as inventors [Attorney Docket No. 3816-10901],
which is a U.S. national stage entry of Patent Cooperation Treaty application no. PCT/US2012/027713, titled “Systems and methods for optimizing medical care through data monitoring and feedback treatment,” filed Mar. 5, 2012, and naming Dimitar V. Baronov, Evan J. Butler, and Jesse M. Lock as inventors [Attorney Docket No. 3816/1009WO],
which claims priority to U.S. provisional patent application No. 61/449,176, filed Mar. 4, 2011.

The disclosure of each of the foregoing is incorporated herein by reference, in its entirety.

### STATEMENT REGARDING FEDERALLY SPONSORED RESEARCH OR DEVELOPMENT

This invention was made with government support under contract number W81XWH-11-C-0086 awarded by the Department of Defense. The government has certain rights in the invention.

### FIELD

Illustrative embodiments generally relate to patient care and, more particularly, various embodiments relate to computer-assisted assessment and treatment.

### BACKGROUND

Practicing medicine is becoming increasingly more complicated due to the introduction of new sensors and treatments. As a result, clinicians are confronted with an avalanche of information, which needs to be evaluated and well understood in order to prescribe a preferred (or potentially optimal) treatment from the multitude of available options, while reducing patient risks. One environment where this avalanche of information has become increasingly problematic is the Intensive Care Unit (ICU). There, the experience of the attending physician and the physician's ability to assimilate the available physiologic information have a strong impact on the clinical outcome. It has been determined that hospitals which do not maintain trained intensivists around the clock experience a 14.4% mortality rate as opposed to a 6.0% rate for fully staffed centers. It is estimated that raising the level of care to that of average trained physicians across all ICUs can save 160,000 lives and $4.3Bn annually. As of 2012, there is a shortage of intensivists, and projections estimate the shortage will only worsen, reaching a level of 35% by 2020.

Therefore, there is a clear need for decision support systems in the ICU which can raise the level of care in facilities which lack trained intensivists.

### BRIEF SUMMARY

Technologies are provided herein for providing a decision support solution to medical professionals to determine medical care through data monitoring and feedback treatment. In one aspect the invention is directed to a system for modeling patient outcomes resulting from treatment in a specific medical area, includes a processor coupled to a memory having computer-executable instructions stored thereon, which when executed by the processor, cause the processor to receive patient-specific data associated with a patient. The system can determine possible patient states for the patient based on the data received, determine a current patient state under which the patient can be categorized, and determine probabilities of the patient transitioning from any of the possible patient states to every other possible patient state.

In another aspect, the invention is directed to a computer-implemented method for modeling patient outcomes resulting from treatment in a specific medical area includes receiving patient-specific data associated with a patient, determining a plurality of possible patient states under which the patient can be categorized, a current patient state under which the patient can be categorized and determining probabilities of the patient transitioning from any of the possible patient states to every other possible patient state.

In yet another aspect, the invention is directed to a computer-readable medium having computer-executable instructions stored thereon, which when executed by a computer, cause the computer to receive patient-specific data associated with a patient, determine possible patient states under which the patient may be categorized and a current patient state under which the patient can be categorized, and determine probabilities of the patient transitioning from any of the possible patient states to every other possible patient state.

### BRIEF DESCRIPTION OF THE DRAWINGS

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

FIG. 1 illustrates a medical care environment for providing health providers assistance in clinical decision making in accordance with various embodiments of the present disclosure;

FIG. 2 illustrates a patient model workflow in accordance with various embodiments of the present disclosure;

FIG. 3 illustrates an exemplary condition network of possible patient states for patients undergoing intensive care after first stage palliation of hypoplastic left heart syndrome in accordance with various embodiments of the present disclosure;

FIG. 4A, FIG. 4B, FIG. 4C and FIG. 4D illustrate a subset of the exemplary condition network of FIG. 3 at various time intervals without exposing the patient to treatment in accordance with various embodiments of the present disclosure;

FIG. 5A, FIG. 5B, and FIG. 5C illustrate a subset of the exemplary condition network of FIG. 3 after exposing a patient to various treatment plans in accordance with various embodiments of the present disclosure;

FIG. 6 is a graph schematically illustrating a sample trajectory of the physiologic variables that can cause a transition from one patient state to another in accordance with various embodiments of the present disclosure; and

FIG. 7 illustrates an exemplary condition network of possible patient states for patients associated with hemorrhaging trauma in accordance with various embodiments of the present disclosure;

FIG. 8 schematically illustrates an exemplary condition network of possible patient states describing cardiogenic shock;

FIG. 9A is a graph schematically illustrating possible patient states of cardiogenic shock, with no medication or device;

FIG. 9B is a graph schematically illustrating possible patient states of cardiogenic shock, with application of a single medication or a single device;

FIG. 9C is a graph schematically illustrating possible patient states of cardiogenic shock, with application of two medications or two devices, or with application of a single medication and a single device;

FIG. 9D is a graph schematically illustrating possible patient states of cardiogenic shock, with application of three medications or three devices, or with application of a single medication and two devices, or with application of two medications and a single device;

FIG. 10A is a flowchart of an embodiment of a method;

FIG. 10B is a flowchart of an embodiment of a method;

FIG. 11 schematically illustrates an embodiment of a graphical user interface;

FIG. 12 schematically illustrates an embodiment of a graphical user interface displaying a plurality of individual graphical user interfaces for a corresponding plurality of individual patients.

### DETAILED DESCRIPTION

Technologies are provided herein for providing a decision support solution to medical professionals to determine medical care through data monitoring and feedback treatment. The technologies described herein can be embodied as a method of determining medical care or as decision support tool configured to operate with real-time monitoring systems that are capable of collecting patient information available from a wide range of sources, such as bedside monitors, lab work, medical records, prescribed treatments, amongst others. This information, along with historical data of similar types of patients, can be used to achieve a paradigm shift from a signal-driven monitoring system to an event-driven monitoring system. That is, instead of the physician being confronted with various physiologic signals and test results, the physician is presented with a qualitative description of the patient's clinical state, the possible clinical states to which the patient may transition, and the probabilities associated with the patient transitioning to each of the possible clinical states from each of the other possible clinical states. The occurrence of a patient transitioning from one possible clinical state to another may be referred to as an event and in an event-driven monitoring system, the physician is focusing on the patient's clinical state as a whole and the possible clinical states to which the patient can transition, instead of focusing on individual signals associated with the multitude of physiological measurements. In this way, the physician may be able to better gauge the risks associated with the patient and formulate a treatment plan based on such risks.

The technologies described herein provide for mathematical models of patient physiology to be merged with expert knowledge of the qualitative behavior of patients in different conditions and under different treatments. The resulting solution allows for the prediction of probable evolutions of the patient's clinical course given the available treatments, and for this information to be presented to physicians in an easily understandable clinical language with which they are comfortable. This also assures that all available information is accounted for by the physicians, independent of their level of training, thereby raising the level of care.

Besides presenting the acquired physiologic information and the consequences of the available treatments in an intuitive way, the technologies described herein enable additional benefits for providing medical care. First, the ability to calculate the probabilities for various possible evolutions of the clinical course enables context dependent alerts. In this case, an alert can be triggered when the probability for a specific adverse event is higher than a pre-specified acceptable threshold. Additionally, acuity metrics can be derived based on the calculated likelihood a patient's condition deteriorates.

Second, the technologies described herein enable the utility of these treatments to be quantified by calculating probable future clinical courses under the various available treatments. As a result, the technologies described herein can assess and determine a treatment and either recommend it to the clinician or render the treatment automatically via the use of infusion pumps, ventilators or any other peripheral medical devices.

The present disclosure will be more completely understood through the following description, which should be read in conjunction with the drawings. In this description, like numbers refer to similar elements within various embodiments of the present disclosure. Within this description, the claims will be explained with respect to embodiments. The skilled artisan will readily appreciate that the methods, apparatus and systems described herein are merely exemplary and that variations can be made without departing from the spirit and scope of the disclosure.

### Definitions

As used in this description and the accompanying claims, the following terms shall have the meanings indicated, unless the context otherwise requires.

The term “clinical risk” means the probability of a patient being in a particular patient state, for example at a particular time.

The term “clinical trajectory” means the sequence of patient states through which a patient evolves during a patient's clinical course.

The term “patient state” means a qualitative description of the physiology of a patient at a particular point of time of the patient's clinical course, which qualitative description is derived from quantified evidence (e.g., measurements of one or more of the patient's internal state variables), and which qualitative description is recognizable by medical practice, and may have implications to clinical decision-making. A patient state may be a medical condition, such as an adverse medical condition, for example. The term “patient state” does not include the patient's state of consciousness (e.g., awake and/or asleep; comatose; conscious; in the process of waking up; in the process of falling asleep; etc.)

Examples of particular patient states include, but are not limited to, adverse medical conditions such as inadequate delivery of oxygen, inadequate ventilation of carbon dioxide, hyperlactatemia, acidosis; cardiogenic shock; amongst others. In addition, these patient states may be specific to a particular medical condition, and the bounds of each of the patient states may be defined by threshold values of various physiological variables and data.

A “treatment regimen” for a patient is a group of treatments for addressing a patient state. The group of treatments may include one or more medications administered to the patient, and or one or more treatments from a treatment device coupled to the patient. In some embodiments, a treatment device may administer a medication. The group of treatments may include not applying any treatment at all (e.g., no medications, no treatments from a set of treatment devices), for example to allow observation of the patient's patient state, and/or changes to or evolution of the patient's clinical trajectory in the absence of treatments.

Sᵢ is a particular patient state that is recognizable by a clinician from collected physiological data. Examples of particular patient states include hypotension with sinus tachycardia, hypoxia with myocardial depression, amongst others.

A particular patient population can exhibit a finite number of possible patient states, Σ={S₁, S₂, S₃, . . . , Sₙ}, in which patients from the patient population can be categorized during their clinical course. Therefore, the clinical course of an individual patient can be described as a sequence of states, SA_{A}→S_{B}S_{C}→S_{D}, where S_{A}, S_{B}, S_{C}, and S_{D} may represent any one of the possible patient states S₁, S₂, S₃, . . . S_{N}. A patient from the particular patient population can only be categorized in only one patient state at any given time. Given that a patient is in a state S₁ the marginal probability that the patient transitions to a new state Sⱼ in a particular time horizon is given by pᵢⱼ.

The treatment applied to a patient can be described by an input vector U={b₁, b₂, . . . , bₖ, d₁, d₂, . . . , dₗ}, which contains effect site medication concentrations B={b₁, b₂, . . . bₖ} (as a non-limiting example, for cardiac medications, the effect site may be the myocardium), and inputs from bedside medical devices D={d₁, d₂, . . . , dₗ} (as a non-limiting example, ventilators, extracorporeal membrane oxygenation machine, heaters, dialysis machine, and others).

It is assumed that the patient physiology is completely described by a vector of physiologic variables, Φ={(φ₁, φ₂, . . . , φₘ}, which can be directly measured or estimated from a combination of different physiologic sensors. For example, the physiologic variable Cardiac Output can be estimated by the Fick's equation by sensing mixed venous oxygenation, arterial oxygenation, and oxygen consumption.

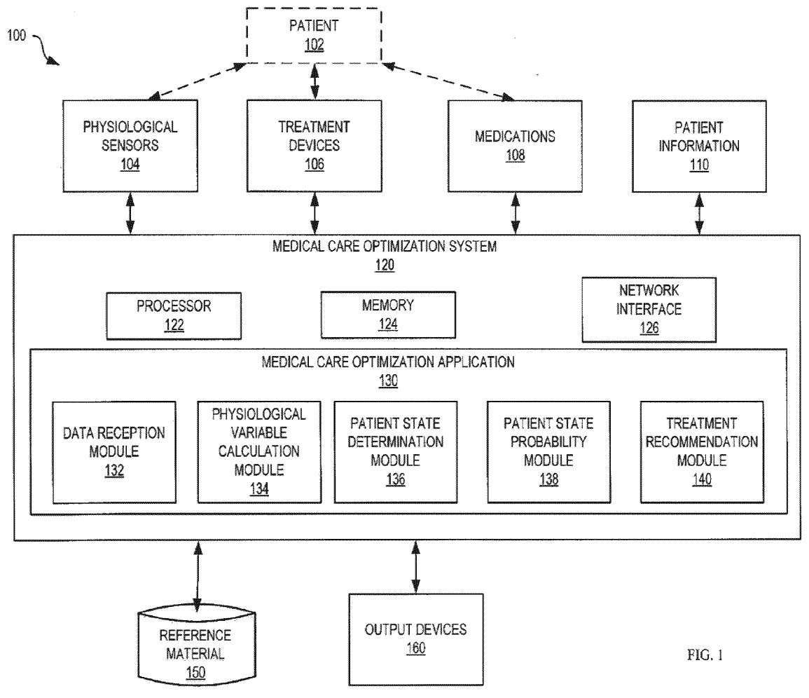

Referring now to the figures, FIG. 1 illustrates a medical care environment 100 for providing health providers, such as physicians, nurses, or other medical care providers, assistance in making clinical decisions about a patient 102 in accordance with various embodiments of the present disclosure. A patient 102 may be coupled to one or more physiological sensors 104 that may monitor various physiological parameters of the patient. These physiological sensors 104 may include but are not limited to, a blood oximeter, a blood pressure measurement device, a pulse measurement device, a glucose measuring device, one or more analyte measuring devices, an electrocardiogram recording device, amongst others. In addition, the patient may be coupled to one or more treatment devices 106 that are configured to administer treatments to the patient 102. In various embodiments, the treatments 106 may be administered in one or more ways, including but not limited to oral, intravenous, and topical medications, therapy, exposure, amongst others. In addition, the patient 102 may further be treated with medications 108, which may also be administered to the patient in one or more ways, including but not limited to orally, intravenously, or topically. By way of the present disclosure, the patient 102 may be afforded improved medical care over existing methods. A medical care system 120, generally referred to herein as the system 120, may be configured to receive patient related information, including real-time information related to the patient's physiology, treatments being provided to the patient, medications being administered to the patient, and other patient related information 110, which may include the patient's medical history, previous treatment plans, results from previous and present lab work, allergy information, predispositions to various conditions, and any other information that may be deemed relevant to make informed decisions regarding the patient's condition and risks, or any combination thereof. For the sake of simplicity, the various types of information listed above will generally be referred to hereinafter as “patient-specific information”. In addition, the system 120 may be configured to utilize the received information, determine possible patient states, determine a patient state from the possible patient states in which the patient is currently categorized, determine the probabilities of transitioning into each of the possible patient states, as well as determine various treatment options and the risks associated with such treatment options, which can then be presented to a medical care provider, including but not limited to a physician, nurse, or other type of clinician.

The system 120, in various embodiments, includes one or more of the following: a computer processor 122, such as a microprocessor available from Intel Corp. for example, a memory 124 coupled to the processor 122, and a network interface 126 configured to enable the system 120 to communicate with other devices over a network. In addition, the system 120 may include a medical care application 130 that may include computer-executable instructions, which when executed by the processor 122, cause the system 120 to be able to afford improved medical care to patients, such as the patient 102.

The medical care application 130 includes, for example, a data reception module 132, a physiological variable estimation module 134, a patient state determination module 136, a patient state probability module 138, and a treatment recommendation module 140 or any combination of the above. In an exemplary embodiment, the data reception module 132 may be configured to receive physiological data from the physiological sensors 104, treatment administration information from the treatment devices 106, medication administering information, and other patient related information, including information collected from the medical devices 104, treatment information from treatments 106, and any other information that may be deemed relevant to make informed decisions regarding the patient's condition and risks, and any combination thereof of the preceding elements. Treatment information may be defined as any information that is related to any treatment that is or has been rendered to a patient.

The physiological variable estimation module 134 may, for example, be configured to utilize the information received by the data reception module 132 and estimate various physiological variables based on the information received. For instance, the variable oxygen delivery cannot be measured through a physiological sensor, but is determined by measuring cardiac output. Possible methods of measuring cardiac output, include but are not limited to, direct measurement through thermodilution, or indirect estimation by substituting mixed venous oxygen content, arterial oxygen content, and oxygen consumption in the Fick equation. It should be appreciated that physiological variables also include physiological variable that can be directly measured by one or more physiologic sensors.

The patient state determination module 136 may, for example, be configured to determine the possible patient states under which the patient may be categorized. Examples of particular patient states include hypotension with sinus tachycardia, hypoxia with myocardial depression, compensated circulatory shock, cardiac arrest, hemorhage, amongst others. In addition, these patient states may be specific to a particular medical condition, and the bounds of each of the patient states may be defined by threshold values of various physiological variables and data. In various embodiments, patient state determination module 136 may determine all possible patient states using one or more of the following: information gathered from reference materials, information provided by health care providers, physiological data of the patient, other patient-specific information, amongst others. The references materials may be stored in a database 150 or other storage device that is accessible to the medical care application 130. These reference materials may include material synthesized from reference books, medical literature, surveys of experts, physician provided information, and any other material that may be used as a reference for providing medical care to patients. In some embodiments, the patient state determination module 136 may first identify a patient population that is similar to the patient. By doing so, the patient state determination module 136 may be able to use relevant historical data based on the identified patient population to determine the possible patient states.

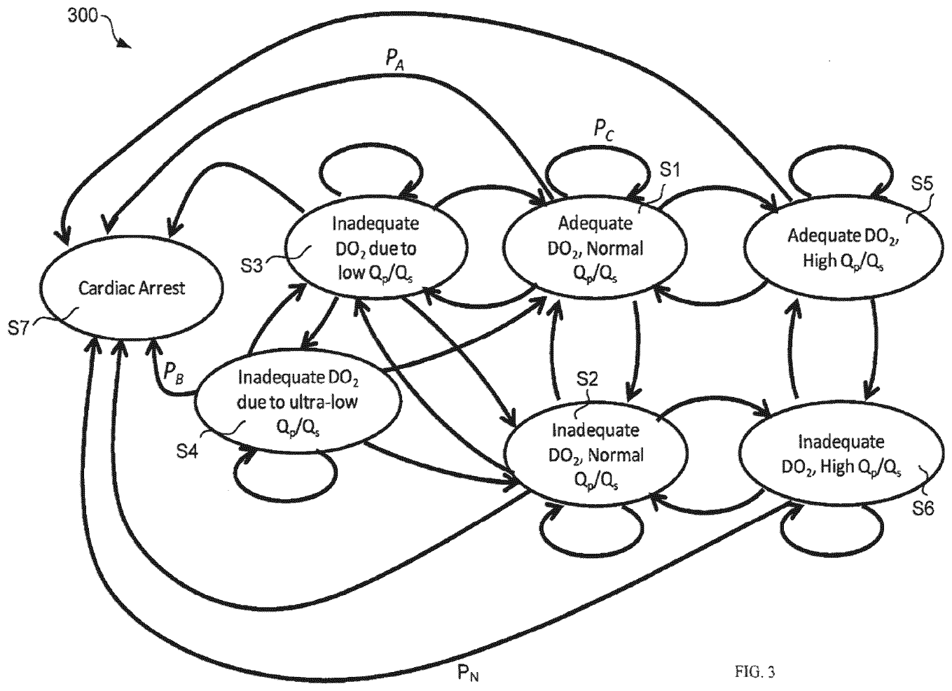

The patient state determination module 136 is capable of also determining the patient state under which the patient is currently categorized, referred to herein as the current patient state. The current patient state of the patient can be determined by analyzing, amongst other things, recent patient-specific information from the patient, including but not limited to real-time physiological data. In some embodiments, the patient state determination module 136 can determine all possible patient states for a patient population and can determine the current patient state of the patient. Additional details related to the patient state determination module 136 will be provided below during a discussion of FIGS. 3-7.

Once the patient state determination module 136 determines the possible patient states under which the patient can be categorized, the patient state probability module 138 is able to determine probabilities associated with the patient transitioning from any patient state to any other patient state or remaining in any particular patient state. The patient state probability module 138 may do so by analyzing the patient-specific information, analyzing historical evidence generated from other patients' patient-specific information, and other information available from the reference material 150. In addition, the patient state probability module 138 may also utilize information received from physicians, medical professionals, scientists, and the like to provide hypothetical risk assessments on patients with particular patient profiles. This information can then be generalized and applied algorithmically to determine the probabilities associated with the patient transitioning from one patient state to any other patient state or remaining in a particular patient state. Additional details related to the patient state probability module 138 will also be provided below during a discussion of FIGS. 3-7.

In various embodiments, if the patient's physiology is changing, either due to treatment being received, or due to the natural changes in the patient's physiology over time, the patient state probability module 138 may be configured to determine updated probabilities of a patient transitioning from one patient state to any other patient state based on the changes in the patient's physiology, or based on other information being provided that may influence the probabilities associated with transitions between the patient states. In some embodiments, the patient state probability module 138 may be configured to determine hypothetical updated probabilities of a patient transitioning from one patient state to any other patient state based on hypothetical assumptions. For example, to determine hypothetical probabilities of a patient transitioning from one patient state to another patient state based on providing a hypothetical treatment, the patient state probability module 138 may utilize historical data to hypothesize how the patient's physiology will change over time based on rendering a particular treatment option to the patient. The patient state probability module 138 may then determine probabilities associated with rendering the hypothetical treatment using the hypothesized changes in patient physiology.

Based on the probabilities determined for each possible transition between patient states, the treatment recommendation module 140 may be configured to provide treatment recommendations. Treatment recommendations are treatment options that may be provided to a patient to improve, for example, the patient's health, quality of life, optimize the cost of care, and other resources, or any combination thereof. In various embodiments, the treatment recommendations may be provided to a health care provider via one or more output devices 160. These output devices include but are not limited to, display units (e.g., computer display devices, such as a computer screen, virtual reality headset, etc.), audio output devices, a printer, or any combination thereof. The treatment recommendation module 140 may also utilize information stored in the reference material 150, and alone or in combination with the patient-specific information, and the probabilities determined for each possible transition between patient states, determine one or more treatment options. Upon determining the treatment options, the treatment recommendation module 140 may be configured to determine which of the treatments appears to be the preferred treatment for the patient at that specific time.

In various embodiments, the treatment recommendation module 140 may be configured to assign a risk index which indicates how likely the patient is to transition from the current patient state to one or more patient states designated as specific morbidity states or a mortality state. Based on this risk index, recommended treatment options may vary. Other types of risks that are considered for determining the recommended treatment include, but are not limited to, morbidity risks, mortality risks, the risks of transitioning into an adverse patient state, the risks associated with transitioning into an improved patient state, and the risks of significantly altering one or more of the physiological variables, risks associated with prolonged hospital stay, or any other risks associated with increased treatment costs to the patient, and the like.

Upon determining the treatment options, the treatment options are then ranked based on the risks described above. The treatment recommendation module 140 may then present, via the output devices 160, the recommended treatment option along with other possible treatment options to the health care provider from which the health care provider can make an informed decision regarding the treatment plan. In some embodiments, the treatment recommendation module may also present additional information, including but not limited to possible complications associated with each treatment option, most likely recovery path and risks associated with the treatment plan. In one embodiment, the treatment recommendation module 140 may be configured to execute the recommended treatment option automatically. As such, the recommended treatment option may send commands to the medical devices and infusion pumps to implement the recommended treatment option, thereby closing the loop between medical sensors and medical treatment.

It should be appreciated that the system is a dynamic system that receives updated patient-specific information periodically. The length of time between receiving updated patient-specific information varies based on the source of the information. Some information may be updated in real-time as it is coming in through a device. In some cases, patient data that is obtained through lab work is updated when the lab work report is entered into the system. The data reception module may provide the information to the remaining modules as the data is received by the data reception module, and the remaining modules may utilize the updated data to perform the functionality associated with the respective modules. This includes updating the current patient state and the probabilities associated with the transitions from each patient state to every other possible patient state upon receiving the updated physiological variable data received.

In various embodiments, the medical care application 130 may include one or more modules that may be configured to perform additional functions. For instance, a context alarm module may be configured to alert the medical provider of changes that may lead to one or more events, including changes in a patient state, changes in risk levels, or probabilities exceeding or falling below threshold values, amongst others. In some embodiments, the medical care application 130 may be configured to automatically alter changes to the treatment being provided to the patient by sending control signals to a particular treatment device 106 causing the treatment device 106 to alter the treatment being provided in accordance with the control signal.

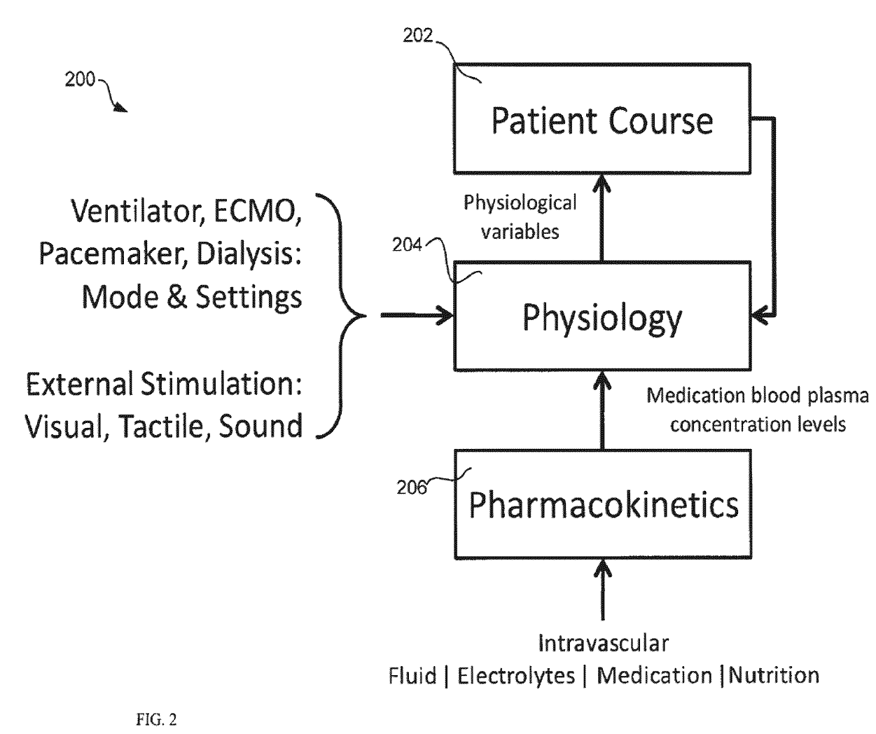

FIG. 2 illustrates a patient model workflow 200 in accordance with various embodiments of the present disclosure. There are three interacting mathematical models within this architecture. The Patient Course block 202 represents the first component, which is modeled as a connected graph describing all possible patient states for any given patient population. Each of these patient states is represented by a node. Connections between nodes represent potential transitions between patient states which occur as the clinical course progresses. The links in the patient states graph are endowed with probabilities indicating the likelihood of each one-step transition. These probabilities, and respectively the patient's clinical course, may be affected by specific medical interventions, which may then be viewed as mechanisms for control. This evokes similarities between the described model and a Markov Decision Process.

The second component is a mathematical model of the patient's underlying physiology 204, referred to hereinafter as physiology model 204. It is assumed that each patient state or groups of patient states can have different mathematical models. The inputs to the physiology model 204 include medication effect site concentrations (i.e. similar to a pharmacodynamic model which abstracts the relationship between the effect site concentration and particular physiologic variables), ventilator settings, which include everything listed in reference to U in the definitions provided above, and other external stimuli. The outputs correspond to the physiologic variables, which in some embodiments, may include arterial blood pressure, systemic or pulmonary resistance, cardiac output, amongst others.

The third component is a pharmacokinetic model 206 which is used to translate medication infusion rates to effect site (e.g. myocardium) concentration levels. It should be appreciated that the pharmacokinetic model 206 may be configured to receive information associated with electrolyte intake, fluid intake, nutritional intake, and medication intake, amongst others.

As shown in FIG. 2, the three mathematical modules connected together form a dynamic system. The dynamic system incorporates a feedback system to account for changes that alter the patient's physiological variables. A patient may exist in a particular patient state based on the current physiological variables of the patient. As the patient undergoes some treatment, for instance, medications being administered to the patient via the pharmacokinetic model 206 alter the patient's physiological variables. Similarly, medical devices coupled to the patient that are also providing treatment of the patient may also alter the treatment being provided to the patient, thereby causing the physiological variables to alter even more. As such, the physiological model 204 experiences changes, which may lead to a transition from the patient's current patient state to another patient state, or may lead to a change in probabilities associated with the possible patient states, which alters the graph of the patient course block 202. Over time, one or more of the patient's physiological variables are continuously changing, thereby altering the probabilities associated with transitioning to other states. This continuous change results in a real-time dynamic system that allows health care providers to render improved medical care to patients.

The following illustrates how the described invention can be applied to the modeling of the clinical course of a specific patient population under intensive care-post-operatively recovering Hypoplastic Left Heart Syndrome patients after stage one palliation.

Hypoplastic Left Hear Syndrome is a congenital heart defect, which is manifested by an underdeveloped left ventricle and left atrium. As a result, patients suffering from this condition do not have separated systemic and pulmonary blood flows, but instead the right ventricle is responsible for pumping blood to both the body and the lungs. Therefore, the hemodynamic management during intensive care involves managing the fractions of the blood flow that pass through the lungs (pulmonary flow Qₚ) and the body (systemic flow Qₛ). The preferred (and perhaps optimal) hemodynamic is reached when, adequate tissue oxygen delivery, DO₂, is achieved for a pulmonary to systemic blood flow ratio, denoted Qₚ/Qₛ, of 1. Often to reach this state the patient physiology passes through other less beneficial states, and the correct identification of these states and the application of proper treatment strategy for each one of them define the quality of the post-operative care. The collection of all these states constitutes the condition network describing this specific population.

FIG. 3 illustrates an exemplary condition network 300 of possible patient states for patients undergoing intensive care after first stage palliation of hypoplastic left heart syndrome in accordance with various embodiments of the present disclosure. It should be appreciated that although these states may not include all possible states in a real-life setting, the following states have been shown for the sake of simplicity and explanation. Additional information regarding these patient states can be found in Moss and Adams' heart disease in infants, children, and adolescents: including the fetus and young adult, Volume 1 (7th ed., pp. 1005-1038). Patient state S₁ refers to Adequate DO₂, Normal Qₚ/Qₛ—This is the preferred state, in which good tissue oxygen perfusion is achieved with minimum work of the heart. A patient in this state is usually weaned from medication and other treatment support.

Patient state S2 refers to Inadequate DO₂, Normal Qₚ/Qₛ—In this state, the patient has preferred (and potentially optimized) pulmonary to systemic flow, but not sufficient tissue oxygenation. This is due to inadequate total cardiac output, which is given by CO=Qₚ+Qₛ. A possible treatment in this case is the administration of chronotropic medications, which can raise the heart rate and respectively the total cardiac output.

Patient state S3 refers to Inadequate DO₂ due to low Qₚ/Qₛ—In this case, the systemic oxygen delivery is prohibited by the fact that there is not enough blood flow oxygenating through the lungs. This can be corrected by raising the systemic vascular resistance with vasoconstrictor medications, re-directing flow towards the lungs.

Patient state S4 refers to Inadequate DO₂ due to ultra-low Qₚ/Qₛ—In this case, even smaller fraction of the blood flow passes through the lungs, e.g. only ⅓ of the total cardiac output is oxygenated. In this extreme case, in addition to increasing systemic vascular resistance, the clinician should consider reducing the pulmonary vascular resistance by administering Nitric Oxide. Alternative, more invasive treatment is to further restrict the shunt through surgical means.

Patient state S5 refers to Adequate DO₂, High Qₚ/Qₛ—In this case, although the body is receiving adequate oxygenation, this is achieved in the expense of increased work of the heart. To correct for this, the clinician should lower systemic vascular resistance either through vasodilator medications or through additional sedation.

Patient state S6 refers to Inadequate DO₂, High Qₚ/Qₛ—In this case, both the tissue oxygenation is insufficient and the pulmonary to systemic blood flow unbalanced. This should be treated by an increase of cardiac output (e.g. chronotropic medication to increase heart rate) and by decrease of systemic vascular resistance.

Patient state S7 refers to cardiac arrest, which represents a major adverse event from untreated inadequate oxygenation or overworked heart, resulting in a cardiac arrest.

Still referring to FIG. 3, the probabilities P_{A} and P_{B} signify, respectively, the probabilities for the patient developing a cardiac arrest in a particular time interval, given adequate oxygen delivery and pulmonary to systemic blood flow ratio close to one, and given compromised hemodynamic with small pulmonary to systemic flow ratio. These probabilities can be calculated by the patient state probability module 138. It should be appreciated that P_{A} should be much smaller than P_{B}, i.e. given preferred (an potentially optimized) hemodynamic, there is a much smaller probability for the patient encountering cardiac arrest. On the other hand, Pc signifies the probability that the patient remains in the same state within the same time interval, i.e. the probability that the patient remains with that (e.g., potentially optimal) hemodynamic. Similarly, all arches from the figure can be endowed with probabilities ranging from 0 to 1 or 0% to 100%.

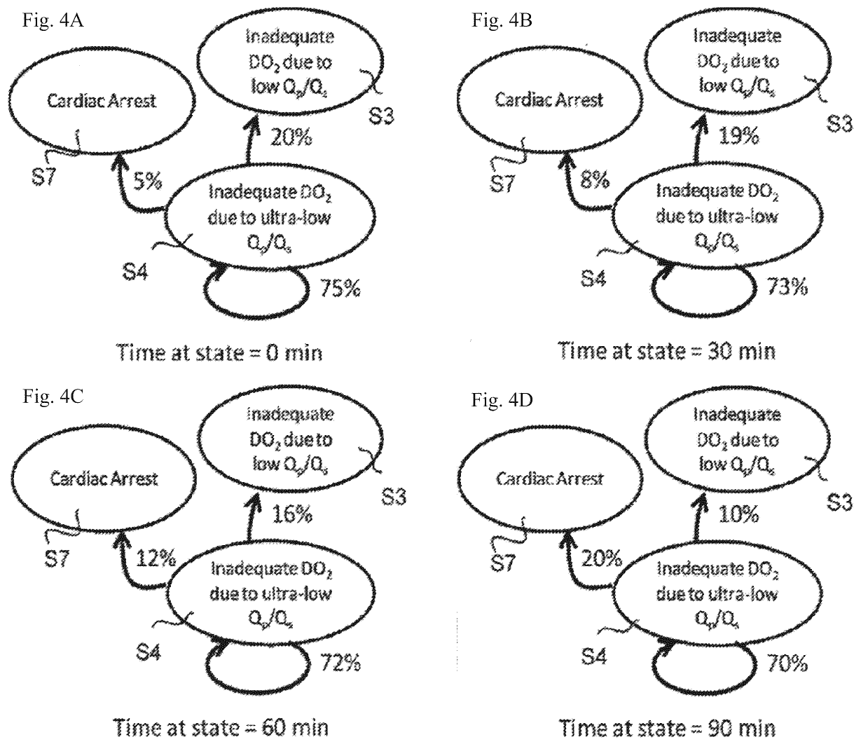

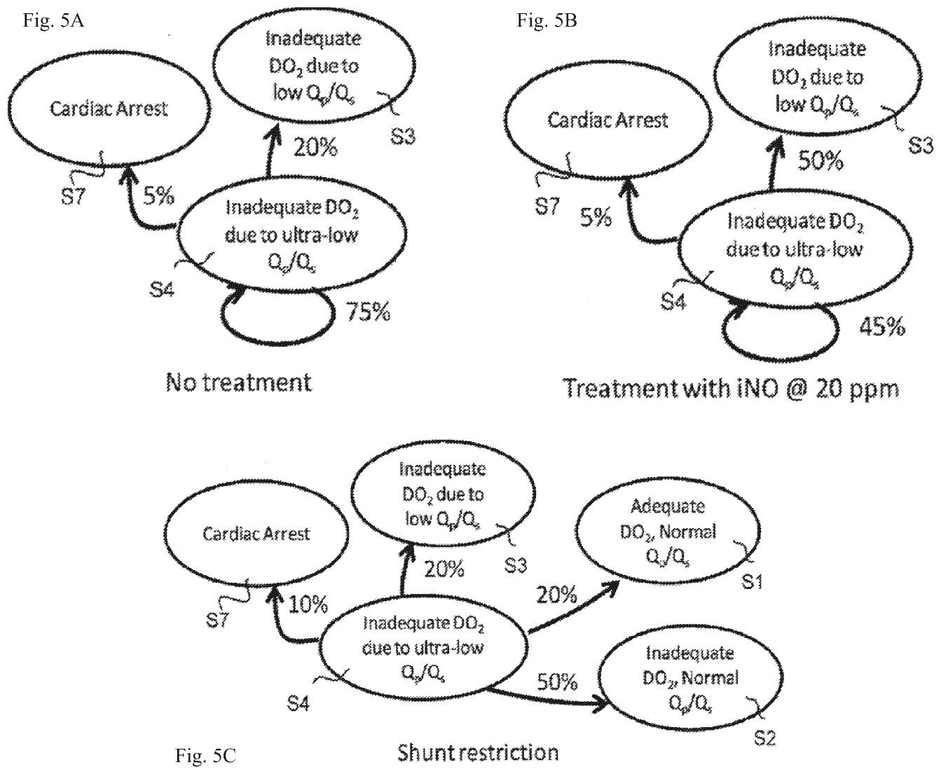

When the system 120 is initialized and no treatment has begun to be administered, the probabilities may be derived from static information, such as medical records, literature, physician inputs, and the like. However, as patient-specific information, such as physiological information and treatment information are provided, the probabilities may be updated based on the patient-specific information being fed to the system 120. The probabilities may be dynamically updated by estimating how the patient is influenced by additional parameters, such as time, as described below with respect to FIGS. 4A, 4B, 4C and 4D, and treatments, as described below with respect to FIGS. 5A, 5B and 5C. As described above, the patient state probability determination module 138 may be configured to estimate the probabilities and update the probabilities as changes to the patient's physiology are observed.

FIGS. 4A, 4B, 4C and 4D illustrate a subset of the exemplary condition network 300 of FIG. 3 at various time intervals without exposing the patient to treatment in accordance with various embodiments of the present disclosure. The simplest influence is the time the patient spends at a particular state, such as patient state S4. After the patient transitions to the patient state S4 with inadequate DO₂ due to ultra-low pulmonary to systemic flow ratio, the chances that the patient transitions to a cardiac arrest state increases with time. This is due to the gradual exhaustion of the patient metabolic reserves, consequent acidosis and hypoxia, which increase the chances for cardiac arrest.

FIGS. 5A, 5B and 5C illustrate a subset of the exemplary condition network 300 of FIG. 3 after exposing a patient to various treatment plans in accordance with various embodiments of the present disclosure. As described above, another way that the transition probabilities can be influenced is through administering treatment. When a patient is in patient state S3, representing an inadequate DO₂ State due to ultra-low pulmonary to systemic flow ratio, there are several treatment options. If nothing is done, the patient will remain in the same state with high probability as indicated in FIG. 5A. If the patient is administered Nitric Oxide, as represented by FIG. 5B, the pulmonary vascular resistance will decrease and therefore there is high probability of transition to a state closer to optimal hemodynamic properties.

Another treatment option is to surgically alter the apex of the shunt, which is represented by FIG. 5C. Although this procedure may have a higher probability for cardiac arrest, there is a zero probability that the patient remains in the same state after it, and high probabilities that the patient transitions to more beneficial hemodynamic states.

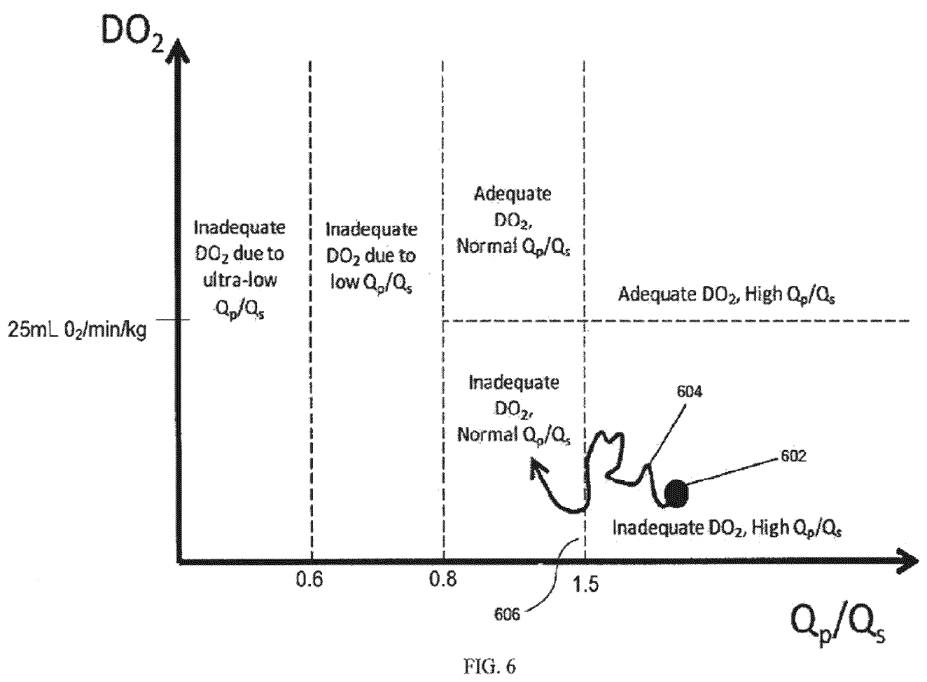

Referring now to FIG. 6, a graph 600 illustrating a sample trajectory of the physiologic variables that can cause a transition from one patient state to another in accordance with various embodiments of the present disclosure is shown. In some embodiments, the probabilities can be further refined and made into functions of measurable physiologic parameters in the following way: first assume that the guiding variables DO₂ and Qₚ/Qₛ form a state space that can be partitioned in the same way as shown in FIG. 6. Assume that for each possible state of the patient there is a set of dynamic equations of the type:

$$x = {f}_{{s}_{i}} ( x , u , μ )$$

$$[ \begin{matrix}{DO}_{2} \\ {Q}_{p} / {Q}_{s}\end{matrix} ] = h ( x )$$

where f and h are known functions, x is a set of internal state variables describing the patient physiology, u is the set of treatment inputs, and is a random variable underscoring the stochastic nature of the dynamics.

Then, assuming that x can be measured at some time t=0, shown as the initial condition 602 in FIG. 5), possible trajectories x(t) 604 can be simulated for different realizations of the random variable and the probabilities for various transitions can be calculated. The transitions can be shown in FIG. 6 as the trajectory crosses the boundaries of the partition of the state space 606.

Consider the following example dynamic equation for the patient being in patient state S6 represented by an Inadequate DO₂, High Qₚ/Qₛ State, and receiving a treatment of chronotropic medication and vasodilators:

$$\frac{d}{d t} [ \begin{matrix}{x}_{1} \\ {x}_{2}\end{matrix} ] = [ \begin{matrix}a ( {x}_{1} - {CO}_{d} ( {u}_{1} ) ) \\ b ( {x}_{2} - \frac{{SVR}_{d} ( {u}_{2} )}{PVR} )\end{matrix} ] + μ ( t )$$

$$[ \begin{matrix}{DO}_{2} ( t ) \\ {Q}_{p} / {Q}_{s} ( t )\end{matrix} ] = [ \begin{matrix}\frac{1}{1 + {x}_{2} ( t )} {x}_{1} ( t ) {Cpv O}_{2} - \frac{1}{{x}_{2} ( t )} {Cv O}_{2} \\ {x}_{2} ( t )\end{matrix} ]$$

where:
x₁ cardiac output defined as x₁=Qₚ+Qₛ
x₂ is the pulmonary to systemic blood flow ratio, Qₚ/Qₛ
a is known constant
b is known constant
CO_{d} is desired cardiac output which is function of the target site concentration of chronotropes uₗ.
PVR is pulmonary vascular resistance
SVR_{d} is desired systemic vascular resistance which is function of the target site concentration of vasodilatation drugs, u₂.
μ(t) is vector Gaussian white noise with known parameters.
CₚᵥO₂ is pulmonary venous oxygen content which is assumed to be known and constant.
CᵥO₂ is the oxygen consumption which is also known,
u1 is the target site concentration of chronotropes
u2 is the target site concentration of vasodilation medications

The equation set forth above has been reported to be used to derive DO₂ as function of x₁, x₂, CₚᵥO₂ and CᵥO₂ is cited by Barnea, O., Santamore, W. P., Rossi, A., Salloum, E., Chien, S., & Austin, E. H. (1998), “Estimation of oxygen delivery in newborns with a univentricular circulation”. Circulation, 98(14), 1407-1413.

Using this equation, the treatment recommendation module 140 of the medical care system 120 can automatically calculate the probabilities of various patient state transitions, given various medication concentrations (u₁,u₂) and given the patient current cardiac output and pulmonary to systemic flow ratio (x₁(0),x₂(O)).

The following illustrates another example of how the described invention can be applied to pre-hospital care of hemorrhaging trauma victims. In the pre-hospital care of this patient population, there are two major treatment choices differentiating the outcome:

The first treatment choice is the choice of a center where the patient should be driven. In this choice, there is a trade-off between the distance to the center and the center capabilities. Ideally all patients with significant injuries will be delivered to a Trauma I level center, which has optimal capabilities. However, especially in a rural set-up, delivering the patient alive to such a center may not be possible, and therefore the patient may have to be triaged to a non-trauma center. The second treatment choice is the infusion of resuscitative fluid. It has been increasingly appreciated by the medical community that the choice of whether fluid infusion should be initiated and the choice of infusion rate should depend on the transportation time. If the patient is close to a hospital no infusion therapy is recommended. As such, by way of the present disclosure, a health care provider debating these treatment choices can make a more informed decision, which results in urging the level of health care being provided to patients towards optimal.

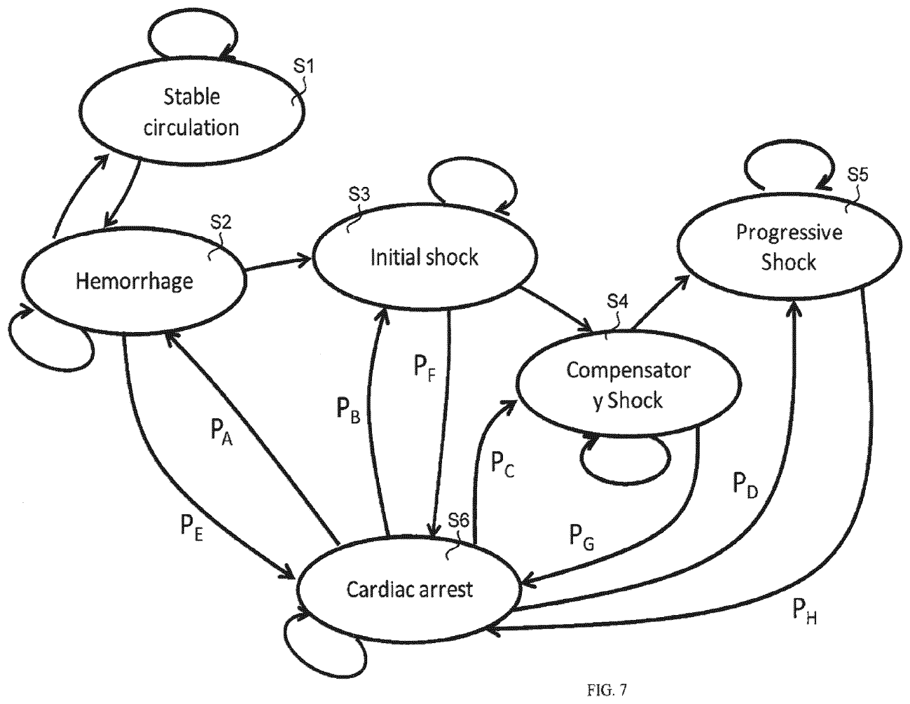

FIG. 7 illustrates an exemplary condition network 700 of possible patient states for patients associated with hemorrhaging trauma in accordance with various embodiments of the present disclosure. It should be appreciated that although these states may not include all possible states in a real-life setting, the following states have been shown for the sake of simplicity and explanation. The major variable guiding the objective identification of the states illustrated in FIG. 7 is the cumulative oxygen deficit (COD). It is defined as the integrated difference between the nominal oxygen consumption (NvO₂), and the oxygen consumption (vO₂) that can be accommodated by the increasingly depleting oxygen caring capacity of the blood:

$$COD = {\int }_{0}^{T} \frac{max [ 0 , {Nv O}_{2} - {v O}_{2} ]}{W} t$$

where W is the weight of the patient.

Still referring to FIG. 7 the following states are illustrated in the condition network 700:

Patient state S1 refers to stable circulation. In this case, homeostasis has been achieved and the remaining blood can assure nominal oxygen consumption.

Patient state S2 refers to hemorrhaging with uncompromised oxygen consumption. In this state, although the patient is losing blood, the patient fluids still have enough oxygen carrying capacity to assure normal oxygen balance.

Patient state S3 refers to initial shock. This state can be quantitatively identified as the COD greater than 0. At this point, the oxygen balance is compromised and the anaerobic metabolism is initiated.

Patient state S4 refers to compensatory shock. This state may, for example, be quantitatively identified as COD between 50 to 120 mL/kg. When in compensatory shock, the physiology of the patient tries to compensate for the compromised oxygen balance by controlling different physiologic variables, such as increasing heart rate to assure sufficient cardiac output, peripheral vasorestriction to assure perfusion of vital organs, increased respiratory rate to counter the ensuing acidosis by faster removal of CO₂, etc.

Patient state S5 refers to progressive shock. This state may, for example, be quantitatively identified as COD greater than 120 mL/kg. At this point, the compensatory mechanisms of the physiology start failing, which leads to consequent failure of vital organs.

Patient state S6 refers to cardiac arrest, which similarly to the previous example is the major adverse effect of the compromised oxygen balance.

Still referring to FIG. 7, the probabilities P_{E}, P_{F}, P_{G} and P_{H} signify, respectively, the probabilities for the patient developing a cardiac arrest in a particular time interval, given hemorrhaging, initial shock, compensatory shock and progressive shock. The patient state probability module 138 can calculate these probabilities. It should be appreciated that P_{H}>P_{G}>P_{F}>P_{E}, i.e. the probability for cardiac arrest grows as the oxygen deficit increases. On the other hand, P_{A}, P_{B}, P_{C}, and P_{D} signify the probabilities of return to spontaneous circulation after cardiac arrest. Again it should be appreciated that P_{A}>P_{B}>P_{C}>P_{D}, i.e. that the higher the oxygen deficit, the lower is the probability that the patient's heart rate returns to normal rhythm. Similar to FIG. 3, all arches shown in FIG. 7 can be endowed with probabilities ranging from, for example, 0 to 1 or 0% to 100%.

When the system 120 is initialized and no treatment has begun to be administered, the probabilities may be derived from static information, such as medical records, literature, physician inputs, and the like. However, as patient-specific information, such as physiological information and treatment information are provided, the probabilities may be updated based on the patient-specific information being fed to the system 120. As described above, the patient state probability determination module 138 may be configured to estimate the probabilities and update the probabilities as changes to the patient's physiology are observed. The probabilities may be dynamically updated by estimating how the patient is influenced by additional parameters, such as the resuscitative fluid infusion rate as described below:

The stated equations below employ the following notation:
x₁ is the cumulative oxygen deficit, COD
x₂ is the hemoglobin also denoted as Hgb
x₃ is the heart's stroke volume
x₄ is the total blood loss
vO₂ is current oxygen consumption
NvO₂ is a constant denoting nominal oxygen consumption
HR is the current heart rate
CO is cardiac output
W is the patient weight
SaO₂ is the arterial oxygen saturation
SvO₂ is the mixed venous oxygen saturation
CaO₂ is the arterial oxygen content
CvO₂ is the mixed venous oxygen content
U is the rate of fluid infusion which is a treatment variable
NV is a constant denoting the nominal volume of blood (the volume of blood before the injury).
cᵢ are constants describing the model, which are specific for each state.
μ_{I} White zero-mean Gaussian noises with different standard deviations.

Then, illustrative dynamic equations describing the evolution of the COD variable outside of the cardiac arrest state can be derived under the following assumptions and dependencies.

$${\dot{x}}_{1} = \frac{{Nv O}_{2} - {v O}_{2}}{W} , \qquad ( 1 )$$

where the oxygen consumption vO₂ can be derived from the Fick principle as:

$${v O}_{2} = CO \cdot ( {CaO}_{2} - {Cv O}_{2} ) = HR \cdot {x}_{3} \cdot 1.36 \cdot {x}_{2} \cdot ( {Sa O}_{2} - {Sv O}_{2} ) .$$

In shock, the heart rate is a compensatory mechanism and can be assumed to be a function of the cumulative oxygen deficit. Therefore,

$$H R = {c}_{1} {x}_{1} + {μ}_{1}$$

Also, SaO₂ can be assumed to be constant equal to 0.99 (or 99% arterial oxygen saturation), and SvO₂ can be assumed to be a function of the oxygen deficit that can be written as:

$${Sv O}_{2} = {c}_{2} - {c}_{3} \frac{{Nv O}_{2} - {v O}_{2}}{W}$$

When substituted back to equation (1) these assumptions and dependencies make \dot{x}₁, a function of the constants cᵢ and the model variables xᵢ.

The next equation shows that the hemoglobin decreases as more fluid is infused to the patient:

$${\dot{x}}_{2} = - {c}_{4} {x}_{2} U + {μ}_{2} . \qquad ( 2 )$$

Similarly the next equation shows that the stroke volume is decreased by the blood loss and increased by the infusion of fluids:

$${\dot{x}}_{4} = - {c}_{5} ( {x}_{4} - U ) + {μ}_{3} . \qquad ( 3 )$$

And the final equation shows that the rate of bleeding is a function of the fluid volume (the more the volume the faster is the bleeding rate):

$${\dot{x}}_{4} = - {c}_{6} ( V - {x}_{4} + U ) + {μ}_{4} . \qquad ( 4 )$$

where the constant c₆ characterizes the injury.

By employing these equations, the system can estimate the infusion rate U that will maximize the probability of the patient being in the least possible harmful state at the estimated time of arrival at the admitting hospital, given the current values of the variables x₁(0), x₂(0), x₃(0), x₄(0).

Moreover, the system can simulate different scenarios of admitting centers and fluid infusion therapies. This will allow the system to identify the risks associate with each of the available triage and fluid therapy options, and identify the least risky pair of an admitting location and fluid therapy.

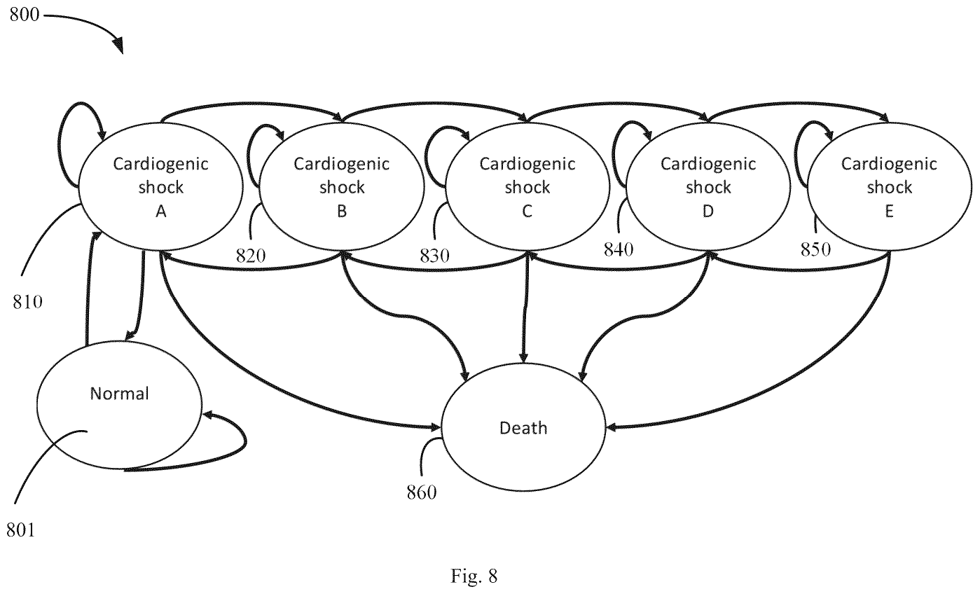

FIG. 8 schematically illustrates an exemplary condition network of possible patient states relating to cardiogenic shock. A “normal” patient state 801 indicates that the patient is not experiencing cardiogenic shock. Patient state 860 (“Death”) indicates that the patient has died or is dead. Patient state “A” 810, patient state “B” 820, patient state “C” 830, patient state “D” 840, and patient state “E” 850 each describe and indicate that the patient is experiencing cardiogenic shock, where each such stage is defined by a combination of patient variables.

The embodiment of FIG. 8 has seven possible patient states, but not all embodiments have seven patient states. An embodiment of possible patient states may have more than seven patient states or fewer than seven patient states.

The patient states have an order, in which patient state of “Normal” 801 is preferable to all other patient states, and patent state “A” is preferable to patient states “B” 820, “C” 830, “D” 840, “E” 850, and “Death” 860. Patient state “B” is preferable to patient states “C” 830, “D” 840, “E” 850, and “Death” 860. Patient state “C” is preferable to patient states “D” 840, “E” 850, and “Death” 860. Patient state “D” is preferable to patient states “E” 850, and “Death” 860. Patient state “E” is preferable to patient state “Death” 860. All patient states are preferable over “Death” 860.

In preferred embodiments, no patient state from the set of possible patient states overlaps another patient state from the set of possible patient states, so that a given combination of systolic blood pressure and lactate level falls exclusively within one of a set of defined patient states.

In preferred embodiments, there is no gap between a patient state and any adjoining patient state, so that every point within a state space falls into one patient state.

In preferred embodiments, every point within a state space falls into one, and only one, patient state. In preferred embodiments, no patient state overlaps any other patient state in the state space.

In illustrative embodiments, a state space is defined by two orthogonal axes, a first axes defined by a first physiological variable from a patient, and a second axis, orthogonal to the first axis, defined by a second physiological variable from the patient. The state space is further divided into a plurality of regions, where each region indicates a corresponding patient state. Each region is defined by a lower SBP boundary and an upper SBP boundary, and a lower lactate boundary and an upper lactate boundary. Each region is distinguished from one or more adjacent regions by its boundaries. In illustrative embodiments, no region overlaps another region, and there is no gap between regions in the state space. A patient with systolic blood pressure and lactate level within a given region is in the patient state corresponding to that region. Consequently, a region may be referred-to as a “patient state.”

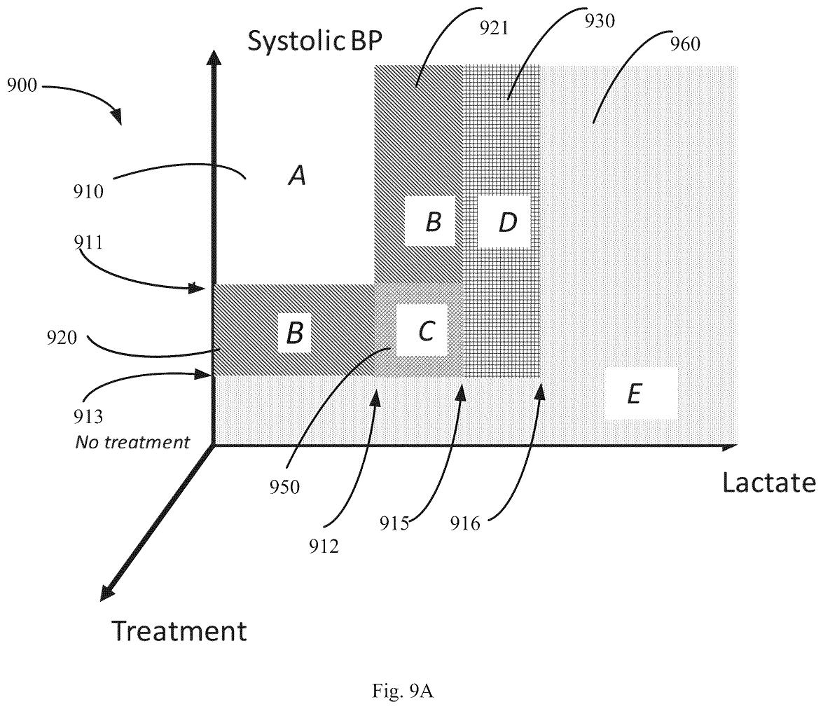

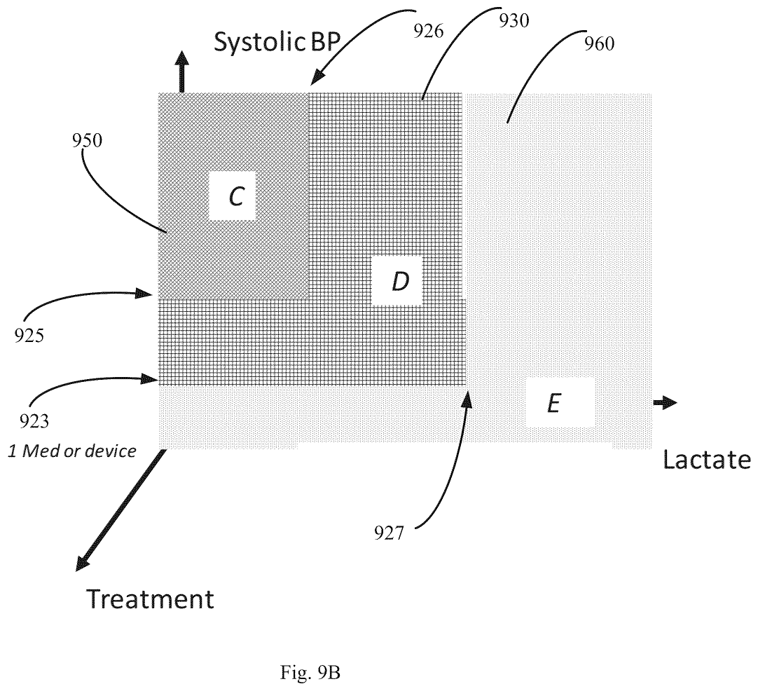

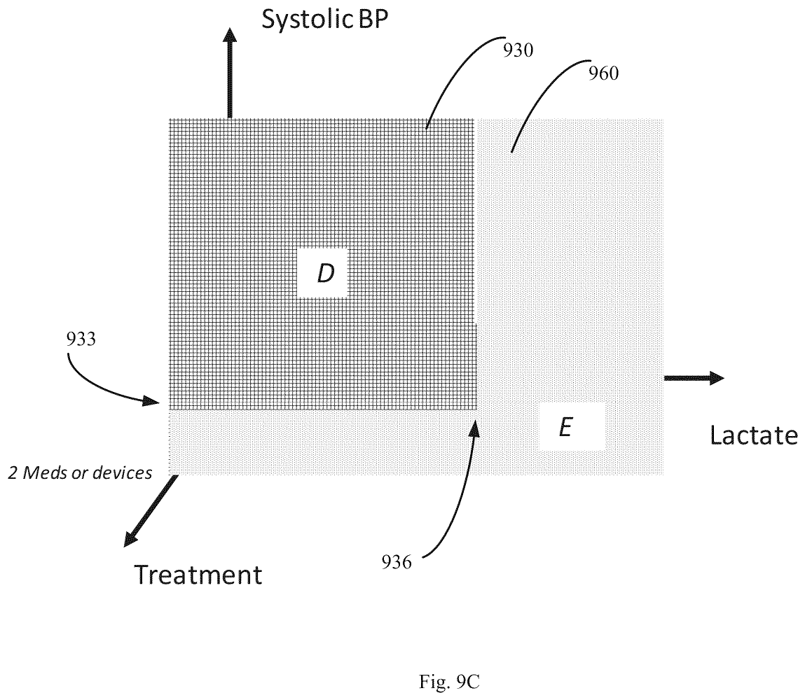

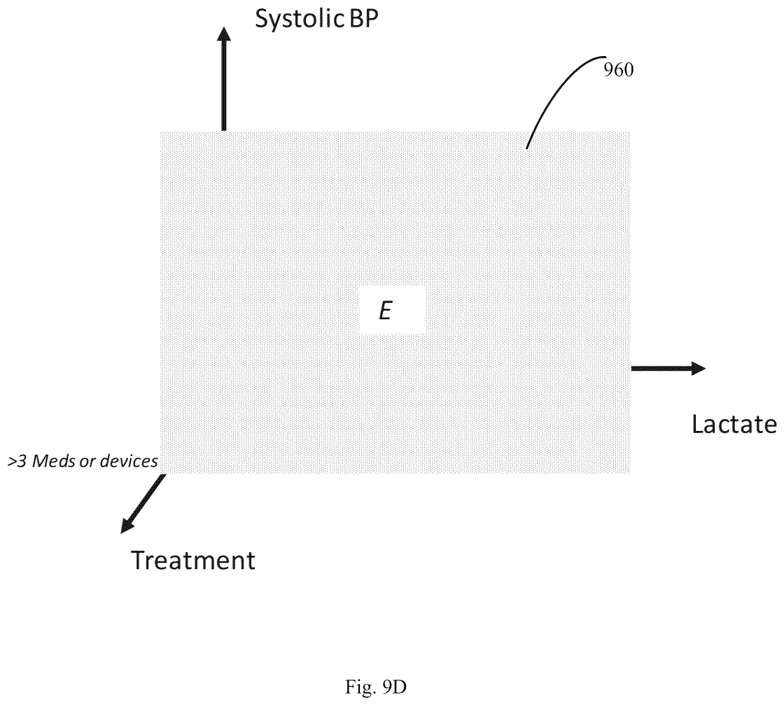

FIG. 9A, FIG. 9B, FIG. 9C and FIG. 9D each schematically illustrates possible patient states of cardiogenic shock. Each patient state is defined by systolic blood pressure and lactate level. The patient's systolic blood pressure may be measured by a blood pressure sensor (one of the sensors 104; such as such as a blood pressure cuff inflated around the patient's upper arm) coupled to the patient and a system 120, and the patient's lactate level may be determined from analysis of a blood sample from the patient and provided to the system 120. The possible patient states can be said to define a state space with systolic blood pressure on a first axis and lactate on a second axis.

Each individual state space is defined by a set of boundaries, including at least one systolic blood pressure (SBP) boundary and at least one lactate (L) boundary. Some embodiments have both a lower SBP boundary and an upper SBP boundary. For some regions of the state space, an SBP boundary may be an upper SBP boundary for one region (or patient state) and the same SBP boundary may be a lower SBP boundary for an adjacent region (or patient state). For some regions of the state space, a lactate (L) boundary may be an upper lactate boundary for one region (or patient state) and the same lactate boundary may be a lower lactate boundary for an adjacent region (or patient state). In illustrative embodiments, a boundary based on a given variable (SBP or lactate level) is a part of a region that has values of that variable that are greater than the boundary.

FIG. 9A is a graph schematically illustrating possible patient states of cardiogenic shock, when the patient is not being treated with any medication and is not being treated by any device.

A first region (or patient state) 910 (which may be referred-to as patient state “A”) is defined by systolic blood pressure above a first systolic blood pressure boundary 911 in combination with a lactate level below a first lactate level boundary 912.

In illustrative embodiments of the first patient state 910, the lactate level 912 is 2 mmol/L, but in other embodiments lactate level of zero mmol/L, one mmol/L or 3 mmol/L.

In illustrative embodiments of the first patient state 910, the first systolic blood pressure level boundary 911 is 90 mmHg, but in other embodiments the first systolic blood pressure level boundary 911 may have a of 85 mmHg or 95 mmHG.

A second region (or patient state) is defined as having two portions: a first portion 920 and a second portion 921 (which together may be referred-to as patient state “B”).

In illustrative embodiments, the first portion 920 has a lower lactate boundary of zero and an upper lactate boundary 912 of 2 mmol/L (but in other embodiments may have a lactate boundary of zero mmol/L, one mmol/L or 3 mmol/L, or within a range between zero and 3 mmol/L), and a systolic blood pressure (“SBP”) with a lower SBP boundary 913 of 60 mmHg (but in other embodiments may have a SBP boundary of 55 mmHg, or 65 mmHg, or within a range of 55 mmHg and 65 mmHg) and an upper SBP boundary 911 of 90 mmHg (but in other embodiments may be 85 mmHg or 95 mmHg, or within a range between 85 mmHg and 95 mmHg). Note that in illustrative embodiments, the upper SBP boundary 911 of region 920 (part of patient state B) is also the lower SBP boundary 911 of region 910 (patient state A) and the upper lactate boundary 912 is also the lower lactate boundary of portion 921 and region 950.

In illustrative embodiments, the second portion 921 (part of patient state B) has a lower boundary lactate level 912 (having value as described above) and an upper boundary lactate level 915 of 6 mmol/L (but in other embodiments may have a lactate boundary of 4 mmol/L, 5 mmol/L or 7 mmol/L, or within a range between 4 mmol/L and 7 mmol/L), along with with a lower SBP boundary 911 (as boundary 911 is described above). In illustrative embodiments, the lower SBP boundary 911 of second region 921 is the same as the lower boundary 911 of region 910 (patient state A).

A third region 950 (which may be referred-to as patient state “C”) is defined as having a lower lactate boundary 912 (having value as described above) and an upper lactate boundary 915 (having value as described above), and a lower SBP boundary 913 (having value as described above) and an upper SBP boundary 911 (having value as described above).

A fourth region 930 (which may be referred-to as patient state “D”) is defined as having a lower lactate boundary 915 (having value as described above) and an upper lactate boundary 916, and a lower SBP boundary 913 (having value as described above). In illustrative embodiments, the upper lactate boundary 916 may be 7 mmol/L, 8 mmol/L, 9 mmol/L, or 10 mmol/L.

A fifth region 960 (which may be referred-to as patient state “E”) has two parts. A first part of region 960 is defined by a lower SBP boundary of zero, and an upper SBP boundary 913 (having value as described above) over a lactate range between zero mmol/L and a first lactate boundary 916 (having value as described above). The second part of region 960 includes all levels of SBP where the lactate is above the first lactate boundary 916. In illustrative embodiments, the first part of region 960 is contiguous with the second part of region 960.

In illustrative embodiments, region 910, region 921, region 930 and region 960 have an upper Systolic Blood Pressure limit defined only by the patient's physiology, and region 960 has an upper lactate limit defined only by the patient's physiology.

In illustrative embodiments, the fourth patient state (D) is a preferred patient state relative to the fifth patient state (E), and the third patient state (C) is a preferred patient state relative to the fourth patient state and the fifth patient state, and the second patient state (B) is a preferred patient state relative to the third patient state and the fourth patient state and the fifth patient state, and the first patient state (A) is a preferred patient state relative to the second patient state and the third patient state and the fourth patient state and the fifth patient state. All patient states are preferred patent states relative to death of the patient 860.

FIG. 9B schematically illustrated possible patient states of cardiogenic shock, with a treatment regimen including application of a single medication or a single device. This embodiment has only three patient states.

A third region (patient state C) 950 is defined as lower lactate level boundary of zero and an upper level lactate boundary 926 of 2 mmol/L, 3 mmol/L, or 4 mmol/L, with systolic blood pressure having a lower SBP boundary 925 of 85 mmHg, or 90 mmHg, or 95 mmHg.

A fourth region (patient state D) 930 is defined as two continuous portions, including a fifth portion having lactate level between zero mmol/L and boundary 927, with systolic blood pressure having an SBP lower boundary 923 of 55 mmHg, 60 mmHg, 65 mmHg, 70 mmHg, 75 mmHg, 80 mmHg, 85 mmHg, 90 mmHg or 95 mmHg. The sixth portion begins at lactate level boundary 226 a systolic blood pressure boundary equal to or greater than boundary 925.

A fifth patient state (E) is defined as two continuous portions, including a third region with lactate level greater than zero mmol/L, with systolic blood pressure having a boundary 923 (with values as described above), and a fourth portion with lactate level boundary 927 (with values as described above) pressure greater than zero mmHg.

In such embodiments, the fourth patient (D) state is a preferred patient state relative to the fifth patient state (E), and the third patient state (C) is a preferred patient state relative to the fourth patient state and the fifth patient state.

FIG. 9C is a graph schematically illustrating possible patient states of cardiogenic shock, with a treatment regimen consisting of (i) a single medication and a single device treatment, (ii) two medications and zero device treatments, or (iii) zero medications two device treatments. This embodiment has only two patient states 930 and 960.

A fourth region (patient state D) is defined having lactate level greater than zero mmol/L, with systolic blood pressure equal to or greater than 55 mmHg. In some embodiments, the fourth patient state (D) is defined having a lower lactate level boundary of than zero mmol/L and an upper lactate level boundary 936 of 8 mmol/L, or 9 mmol/L, or 10 mmol/L or 11 mmol/L, along with systolic blood pressure having a lower SBP boundary 933 of 55 mmHg, 60 mmHg or 65 mmHg.

A fifth region (patient state E) is defined as two contiguous portions, including a third portion with lactate level between zero mmol/L and boundary 936 (with values described above), along with systolic blood pressure having a lower SBP boundary of zero mmHg and an upper DBP boundary 933 (with values as described above), and a fourth region with lactate level lower boundary 936 (with values as described above), with systolic blood pressure greater than zero mmHg.

In such embodiments, the fourth patient (D) state is a preferred patient state relative to the fifth patient state (E).

FIG. 9D is a graph schematically illustrating a possible patient state of cardiogenic shock, with a treatment regimen consisting of application of (i) a single medication and two device treatments, (ii) two medications and a single device treatment, and (iii) three medications and zero device treatments, and (iv) zero medications and three device treatments. This embodiment consists of a single patient state, patient state (E) which is defined lactate level equal to or greater than zero mmol/L, with systolic blood pressure equal to or greater than zero mmHg. In this embodiment, the fifth patient (E) state is a preferred patient state relative to death of the patient.

Other embodiments use different physiological variables, or different combinations of physiological variable, than those described above in connection with FIG. 9A, FIG. 9B, FIG. 9C and FIG. 9D, and FIG. 9E. For example, some embodiments have fewer than the five patient states (A, B, C, D, and E) described above FIG. 9A, FIG. 9B, FIG. 9C and FIG. 9D, and FIG. 9E.

For example, in some embodiments a second patient state (B), with a treatment regimen that omits medications and device treatment, is defined by systolic blood pressure (SBP) between 60-90 mmHg along with mean arterial pressure (MAP) between 50 and 65 mmHg. In some embodiments a second patient state (B), with a treatment regimen that omits medications and device treatment, is defined by lactate level between 2-5 mmol/L or ALT between 200-500 U/L. Mean arterial pressure may be determined using data from a set of pressure sensors coupled to the patient.

In some embodiments, a third patient state (C), with a treatment regimen that omits medications and device treatment, is defined by systolic blood pressure (SBP) between 60-90 mmHg along with mean arterial pressure (MAP) between 50 and 65 mmHg, in conjunction with lactate level between 2-5 mmol/L or ALT between 200-500 U/L.

In some embodiments, a fourth patient state (D), is defined by systolic blood pressure (SBP) between 60-90 mmHg along with mean arterial pressure (MAP) between 50 and 65 mmHg, in conjunction with lactate level between 5-10 mmol/L or ALT greater than 500 U/L. In some embodiments, the fourth patient state (D), is defined by treatment being administered to the patient, the treatment regimen including between two and five medications, or including between two and five device treatments, or between two and five treatments including a combination of medications and device treatments.

In some embodiments, a fifth patient state (E), is defined by systolic blood pressure less than 60 mmHg along with mean arterial pressure (MAP) less than 50 mmHg, or lactate level greater than 10 mmol/L, or pH less than 7.2. In some embodiments, the fifth patient state (E), is defined by a treatment regimen being administered to the patient, the treatment regimen including greater than or equal to three medications, or including greater than or equal to three treatment devices.

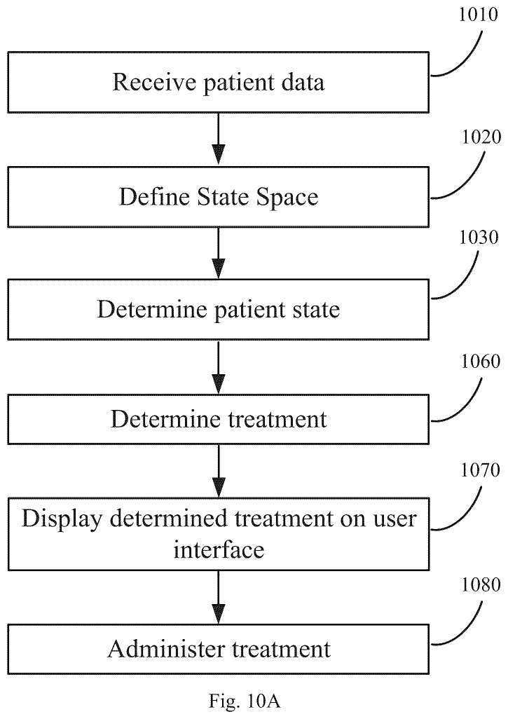

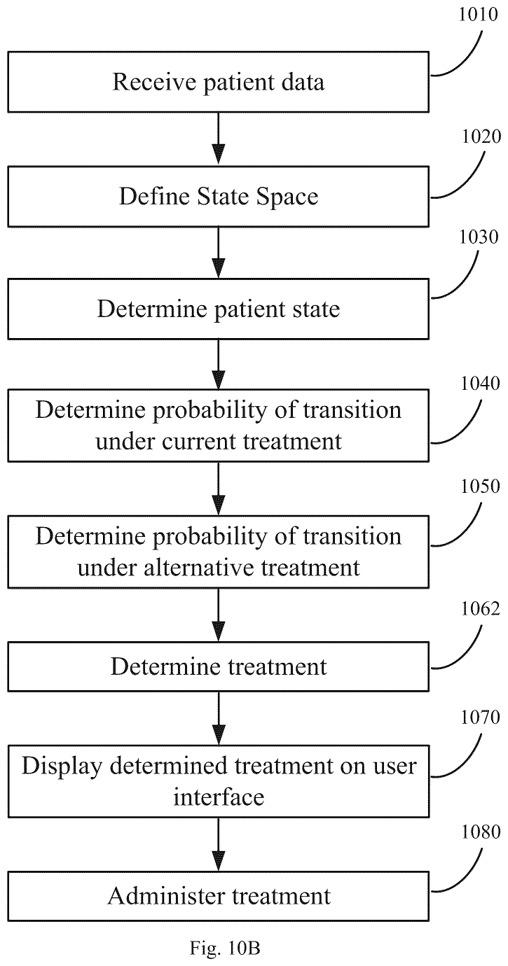

FIG. 10A is a flowchart of an embodiment of a method, which may be a computer-implemented method.

Step 1010 includes, receiving at a computer processer 122, patient-specific data associated with the patient. In illustrative embodiments, the patient-specific data includes at least a first physiological variable and a second physiological variable from the patient.

In some embodiments, the first physiological variable is the patient's lactate level, for example as determined by a laboratory test performed on a blood sample drawn from the patient, and the second physiological variable is the patient's systolic blood pressure as determined, by a non-invasive measurement taken by one or more blood pressure sensors coupled to the patient, such measurement taken from the patient contemporaneously with the blood sample drawn from the patient and used to determine the patient's lactate level.

In some embodiments, the first physiological variable is the patient's Alanine Transaminase Level (“ATL”), for example as determined by a laboratory test performed on a blood sample drawn from the patient, and the second physiological variable is the patient's mean blood pressure as determined, by determining the mathematical mean of a plurality of a non-invasive measurements taken from the patient over time.

Step 1020 includes defining a state space with the first physiological variable on a first axis and the second physiological variable on a second axis, the state space partitioned into a plurality of possible clinical patient states of cardiogenic shock defined by a plurality of boundaries, which boundaries are defined by quantitative points of the first physiological variable and the second physiological variable. In preferred embodiments, no patient state overlaps any other patient state in the state space. In preferred embodiments, there are no gaps between adjacent patient states.

Step 1030 includes determining, from the patient-specific data, a current clinical patient state of the patient within the state space, the current clinical patient state being one of the plurality of possible patient states.

Step 1060 includes determining, or selecting, a selected treatment regimen, based on the current clinical patient state as determined from the state space. In the embodiment of FIG. 10A, each patient state has an associated treatment regimen as shown in Table 1, which associated treatment regimen is stored in a memory in communication with the computer. A treatment regimen associate with each patient state may be prescribed by a treating physician, or determined by literature in the field. The selected treatment regimen may be determined by selecting the treatment associated with the current clinical patient state.

TABLE 1

| Patient State | Treatment Regimen |
|---|---|
| A | Regimen A |
| B | Regimen B |
| C | Regimen C |
| D | Regimen D |
| E | Regimen E |

Treatment for cardiogenic shock may include administering to a patient one or more medications, including one or more of epinephrine; dopamine; norepinephrine; vasopressin and/or milrinone, a vasopressor, or an inotrope. One or more of such medications may be administered to the patient by a treatment device under control of a computer.

Treatments for cardiogenic shock may include administering to a patient support from one or more treatment devices, such support including temporary ventricular assistance (e.g., from a Temporary Ventricular Assist Device); extracorporeal membrane oxygenation (“ECMO”) (e.g., from an extracorporeal membrane oxygenation machine), and/or application of an Intra-aortic balloon pump, to name but a few examples.

Parameters of such machines, controlled by a computer, may include flow rate, pressure, oxygenation, a dose of a medication, timing of administration of treatment, schedule of administration of treatment, and frequency of administration of treatment, to name but a few examples.

Some embodiments include step 1070, which includes causing a display (or displaying), on an electronic computer display device 160, information identifying the selected treatment regimen.

Step 1080 includes sending an electronic signal from the computer to a treatment device coupled to the patient so that the signal causes the treatment device to administer the selected treatment regimen to the patient.

FIG. 10B is a flowchart of an embodiment of a method, which may be a computer-implemented method.

Step 1010, step 1020, step 1030, step 1070 and step 1080 are the same as described in connection with FIG. 10A.

Step 1040 includes determining a set of current probabilities, each current probability of the set of current probabilities defining a probability of the patient transitioning from the current patient state to another patient state of the plurality of possible patient states under the current treatment regimen applied to and received by the patient. Each such current probability may be determined by analysis of historical data of a population of people having the current patient state (i.e., the same patient state as the patient) and undergoing the current treatment (i.e., the same treatment applied to and received by the patient). Such historical data, and/or a listing of transition probabilities, may be taken from original research, or from available scientific literature. Such historical data, and/or a listing of transition probabilities, may be stored in a system memory 124, or in a database of reference material 150, so name but a few examples. The historical data may not be the same for all patients, for example because the historical data may depend on the patient's age; sex; weight; or other health conditions. A person of ordinary skill in the art would be able to specify or identify the historical data for each patient.

The following tables (Table 2A and Table 2B) illustrate an example of such historical data:

TABLE 2A

| | | Probability | Probability | Probability | Probability | Probability |
|---|---|---|---|---|---|---|
| | | of | of | of | of | of |
| | | Transition | Transition | Transition | Transition | Transition |
| Current | Current | to Patient | to Patient | to Patient | to Patient | to Patient |
| Patient | Treatment | State | State | State | State | State |
| State | Regimen | A | B | C | D | E |
| Patient | Treatment | % AA1 | % AB1 | % AC1 | % AD1 | % AE1 |
| State A | 1 | | | | | |
| Patient | Treatment | % AA2 | % AB2 | % AC2 | % AD2 | % AE2 |
| State A | 2 | | | | | |
| Patient | Treatment | % BA1 | % BB1 | % BC1 | % BD1 | % BE1 |
| State B | 1 | | | | | |
| Patient | Treatment | % BA2 | % BB2 | % BC2 | % BD2 | % BE2 |
| State B | 2 | | | | | |
| Patient | Treatment | % CA1 | % CB1 | % CC1 | % CD1 | % CE1 |
| State C | 1 | | | | | |
| Patient | Treatment | % CA2 | % CB2 | % CC2 | % CD2 | % CE2 |
| State C | 2 | | | | | |
| Patient | Treatment | % DA1 | % DB1 | % DC1 | % DD1 | % DE1 |
| State D | 1 | | | | | |
| Patient | Treatment | % DA2 | % DB2 | % DC2 | % DD2 | % DE2 |
| State D | 2 | | | | | |
| Patient | Treatment | % EA1 | % EB1 | % EC1 | % ED1 | % EE1 |
| State E | 1 | | | | | |
| Patient | Treatment | % EA2 | % EB2 | % EC2 | % ED2 | % EE2 |
| State E | 2 | | | | | |

TABLE 2B

| | | Probability of | Probability of |
|---|---|---|---|
| | Current | Transition to | Transition to |
| Current | Treatment | Patient State | Patient State |
| Patient State | Regimen | Normal | Dead |
| Patient State A | Treatment 1 | % AN1 | % AX1 |
| Patient State A | Treatment 2 | % AN2 | % AX2 |
| Patient State B | Treatment 1 | % BN1 | % BX1 |
| Patient State B | Treatment 2 | % BN2 | % BX2 |
| Patient State C | Treatment 1 | % CN1 | % CX1 |
| Patient State C | Treatment 2 | % CN2 | % CX2 |
| Patient State D | Treatment 1 | % DN1 | % DX1 |
| Patient State D | Treatment 2 | % DN2 | % DX2 |
| Patient State E | Treatment 1 | % EN1 | % EX1 |
| Patient State E | Treatment 2 | % EN2 | % EX2 |

Step 1050 includes determining a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment.

Each such alternative probability may be determined by analysis of historical data of a population of people having the current patient state (i.e., the same patient state as the patient) and undergoing the current treatment regimen (i.e., the same treatment regimen applied to and received by the patient). Such historical data, and/or a listing of transition probabilities, may be taken from original research, or from available scientific literature. Such historical data, and/or a listing of transition probabilities, may be stored in a system memory 124, or in a database of reference material 150, so name but a few examples.

The following tables (Table 3A and Table 3B) illustrates an example of such historical data:

TABLE 3A

| | | Probability | Probability | Probability | Probability | Probability |
|---|---|---|---|---|---|---|
| | | of | of | of | of | of |
| | | Transition | Transition | Transition | Transition | Transition |
| Current | Alternative | to Patient | to Patient | to Patient | to Patient | to Patient |
| Patient | Treatment | State | State | State | State | State |
| State | Regimen | A | B | C | D | E |
| Patient | Alt. | % Alt-AA1 | % Alt-AB1 | % Alt-AC1 | % Alt-AD1 | % Alt-AE1 |
| State A | Treatment | | | | | |
| | Regimen 1 | | | | | |
| Patient | Alt. | % Alt-AA2 | % Alt-AB2 | % Alt-AC2 | % Alt-AD2 | % Alt-AE2 |
| State A | Treatment | | | | | |
| | Regimen 2 | | | | | |
| Patient | Alt. | % Alt-BA1 | % Alt-BB1 | % Alt-BC1 | % Alt-BD1 | % Alt-BE1 |
| State B | Treatment | | | | | |
| | Regimen 1 | | | | | |
| Patient | Alt. | % Alt-BA2 | % Alt-BB2 | % Alt-BC2 | % Alt-BD2 | % Alt-BE2 |
| State B | Treatment | | | | | |
| | Regimen 2 | | | | | |
| Patient | Alt. | % Alt-CA1 | % Alt-CB1 | % Alt-CC1 | % Alt-CD1 | % Alt-CE1 |
| State C | Treatment | | | | | |
| | Regimen 1 | | | | | |
| Patient | Alt. | % Alt-CA2 | % Alt-CB2 | % Alt-CC2 | % Alt-CD2 | % Alt-CE2 |
| State C | Treatment | | | | | |
| | Regimen 2 | | | | | |
| Patient | Alt. | % Alt-DA1 | % Alt-DB1 | % Alt-DC1 | % Alt-DD1 | % Alt-DE1 |
| State D | Treatment | | | | | |
| | Regimen 1 | | | | | |
| Patient | Alt. | % Alt-DA2 | % Alt-DB2 | % Alt-DC2 | % Alt-DD2 | % Alt-DE2 |
| State D | Treatment | | | | | |
| | Regimen 2 | | | | | |
| Patient | Alt. | % Alt-EA1 | % Alt-EB1 | % Alt-EC1 | % Alt-ED1 | % Alt-EE1 |
| State E | Treatment | | | | | |
| | Regimen 1 | | | | | |
| Patient | Alt. | % Alt-EA2 | % Alt-EB2 | % Alt-EC2 | % Alt-ED2 | % Alt-EE2 |
| State E | Treatment | | | | | |
| | Regimen 2 | | | | | |

TABLE 3B

| | | Probability of | Probability of |
|---|---|---|---|
| | Current | Transition to | Transition to |
| Current | Treatment | Patient State | Patient State |
| Patient State | Regimen | Normal | Dead |
| Patient State A | Alt. Treatment | % Alt-AN1 | % Alt-AX1 |
| | Regimen 1 | | |
| Patient State A | Alt. Treatment | % Alt-AN2 | % Alt-AX2 |
| | Regimen 2 | | |
| Patient State B | Alt. Treatment | % Alt-BN1 | % Alt-BX1 |
| | Regimen 1 | | |
| Patient State B | Alt. Treatment | % Alt-BN2 | % Alt-BX2 |
| | Regimen 2 | | |
| Patient State C | Alt. Treatment | % Alt-CN1 | % Alt-CX1 |
| | Regimen 1 | | |
| Patient State C | Alt. Treatment | % Alt-CN2 | % Alt-CX2 |
| | Regimen 2 | | |
| Patient State D | Alt. Treatment | % Alt-DN1 | % Alt-DX1 |
| | Regimen 1 | | |
| Patient State D | Alt. Treatment | % Alt-DN2 | % Alt-DX2 |
| | Regimen 2 | | |
| Patient State E | Alt. Treatment | % Alt-EN1 | % Alt-EX1 |
| | Regimen 1 | | |
| Patient State E | Alt. Treatment | % Alt-EN2 | % Alt-EX2 |
| | Regimen 2 | | |

Step 1062 includes determining, or selecting, a selecting, a selected treatment from among the current treatment and the set of alternative treatments, the selected treatment being that treatment most likely, as determined by the set of alternative probabilities, to cause the patient to transition from the current patient state to a preferred patient state. In illustrative embodiments, the preferred patient state is a patient state that is closer to the Normal patient state 801, and further from (or at least not closer to) Death 860, than the current patient state. In illustrative embodiments, the preferred patient state may be the current patient state.

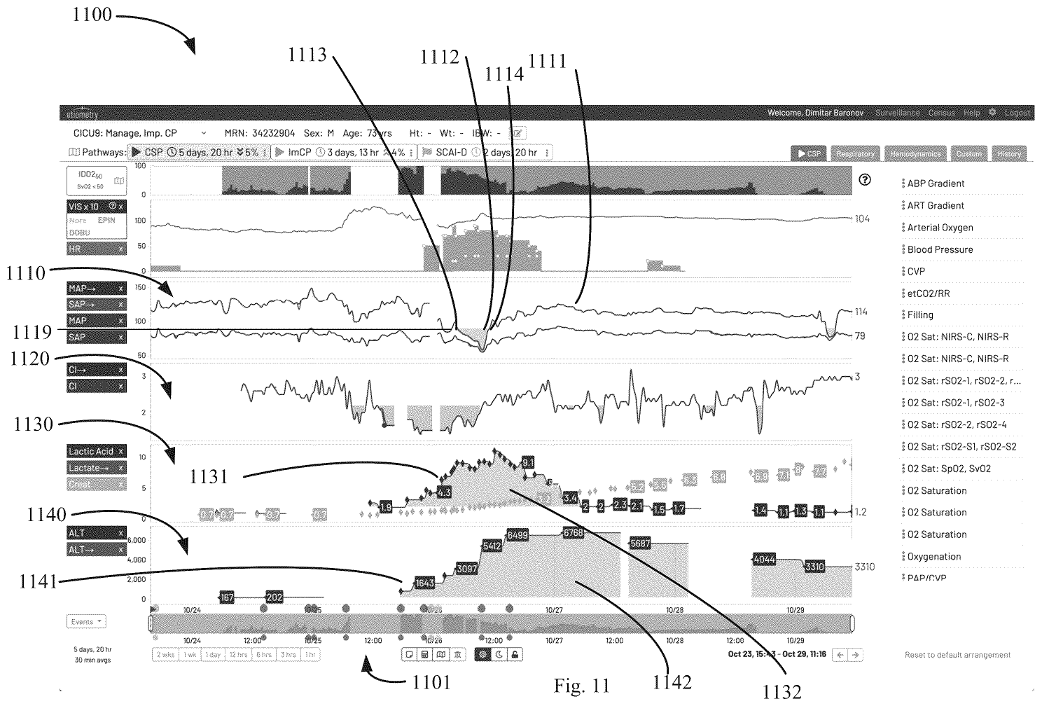

FIG. 11 schematically illustrates an embodiment of a graphical user interface 1100. The graphical user interface 1100 includes a set of windows, each of which graphically displays readings taken from a patient. For example, window 1110 includes a trace 1111 of MAP data taken from a specific patient over time. The trace 1111 represents a plurality of datapoints over time. Illustrative embodiments update the data (e.g., receive updated patient data) of the graphical user interface at a frequency of once per minute, or once every two minutes, to name but a few examples.

Window 1110 also graphically indicates when a trace falls below a threshold, such as a boundary that defines, in part, a patient state or region in a state space. For example, trace 1111 falls below a MAP threshold and remains below that threshold for a period of time. Moreover, the amount by which the MAP trace is below the MAP threshold changes over time. In window 1110, to avoid cluttering the graphical user interface, there is no line across the breadth of the window 1110 graphically showing the MAP threshold. Consequently, the location where the MAP trace 1111 falls below the MAP threshold is difficult for a user to determine visually, and the location where the MAP trace 1111 rises above the MAP threshold is also difficult for a user to determine visually. Further, the depth of the MAP 1111 trace below the MAP threshold is difficult for the user to determine visually.

To address those problems, the graphical user interface 1100 graphically shows the area where the MAP trace 1111 is below the threshold as shaded. In FIG. 11, the threshold is identified by line 1119, although illustrative embodiments do not include such a line in the graphical user interface 1100.

The shaded area visually indicates to the user the point 1113 where the trace 1111 falls below the MAP threshold (1119), as well as the amount by which the MAP trace 1111 is below the MAP threshold (1119) as it changes over time, and the point in time 1114 were the trace rises above the MAP threshold (1119). To generate the window 1110, a computer accesses the data points represented by the trace 1111, and the MAP threshold (represented by line 1119), and compares each data point to the MAP threshold to identify point 1113 in time at which the trace 1111 falls below the MAP threshold, and point 1114 in time at which the trace 1111 next rises above the MAP threshold. Then, for all points between point 1113 and point 1114, the computer identifies the distance (measured in lactate level) between the trace 1111 (i.e., the data points) and the MAP threshold (1119), and shades the area between the trace 1111 and the MAP threshold (1119).

The graphical user interface 1100 is beneficial because it is important for a treating medical professional (e.g., doctor; nurse) to easily and quickly identify when the trace 1111 is below the MAP threshold (1119); the point in time 1113 at which the trace 1111 fell below the MAP threshold (1119); how much the trace 1111 is below the MAP threshold (1119) at any given point in time; the point in time 1114 that the trace 1111 rises above the MAP threshold (1119), and the duration of time that the trace was below the MAP threshold (1119). The shaded area 1112 graphically indicates the area between the trace 1111 and the MAP threshold, thereby graphically indicating to the user the aggregate amount (e.g., the two-dimensional area between the trace 1111 and the MAP threshold 1119) that the patient's MAP level is or was below the MAP threshold. The foregoing information would be difficult, or perhaps impossible, for a human to discern from the trace or other elements of the window 1110 without the shading described above, particularly without a line indicating the threshold (e.g., 1119), and particularly with a level of accuracy required for medical treatment of a patient.

Window 1130 schematically illustrates a trace 1131 of a patient's lactate level. In window 1130, to avoid cluttering the graphical user interface, there is no line across the breadth of the window 1130 graphically showing the lactate threshold. Consequently, the location where the lactate trace 1131 rises above the lactate threshold is difficult for a user to determine visually, and the location where the lactate trace 1131 falls below the lactate threshold is also difficult for a user to determine visually. Further, the height of the lactate trace 1131 above the lactate threshold is difficult for the user to determine visually.

To address those problems, the graphical user interface 1100 graphically shows the area where the lactate trace 1131 is above the lactate threshold as shaded. The shaded area visually indicates to the user the point in time at which the trace 1131 rises above the lactate threshold, as well as the amount by which the trace 1131 exceeds the lactate threshold as it changes over time, and the point in time at which the trace 1131 falls below the lactate threshold. The graphical user interface 1100 is beneficial because it is important for a treating medical professional (e.g., doctor; nurse) to easily and quickly identify when the trace 1131 is above the lactate threshold; when the trace 1131 rose above the lactate threshold; how much the trace 1131 is above the lactate threshold at any given point in time; the point in time that the trace 1131 falls below the lactate threshold, and the duration of time that the trace was above the lactate threshold. The shaded area 1132 graphically indicates the area between the trace 1311 and the lactate threshold, thereby graphically indicating to the user the aggregate amount that the patient's lactate level is or was above the lactate threshold.

Window 1140 schematically illustrates a trace 1141 of a patient's ALT level. The shaded area 1142 graphically and visually indicates to the user the point in time at which the trace 1141 rises above the ALT threshold, as well as the amount by which the trace 1141 exceeds the ALT threshold as it changes over time, and the point in time at which the trace 1141 falls below the ALT threshold. The graphical user interface 1100 is beneficial because it is important for a treating medical professional (e.g., doctor; nurse) to easily and quickly identify when the trace 1141 (representing the patient's ALT level over time) is above the ALT threshold; when the trace 1141 rose above the ALT threshold; how much the trace 1141 is above the ALT threshold at any given point in time; the point in time that the trace 1141 falls below the ALT threshold, and the duration of time that the trace 1141 was above the ALT threshold. The shaded area 1142 graphically indicates the area between the trace 1141 and the lactate threshold, thereby graphically indicating to the user the aggregate amount that the patient's ALT level is or was above the ALT threshold.

According to the foregoing, the graphical user interface 1100 may be described as a graphical user interface having a set of windows 1110; 1120; 1140, each window having a time axis 1101, and a trace (1111; 1121; 1131; 1141) schematically illustrating a patient's patient state variable over time. Each window also includes a shaded area graphically (visually) identifying to a user one or more of the following:
(a) the point in time at which the trace rises above (or below) a threshold for the patient state variable represented by the trace;
(b) the amount by which the trace exceeds (or is below) that threshold as it changes over time;
(c) the point in time at which the trace falls below (or rises above) that threshold; and/or
(d) the area between the trace and the threshold, thereby graphically indicating to the user the aggregate amount that the patient's state variable level is or was above (or below) the associated threshold.

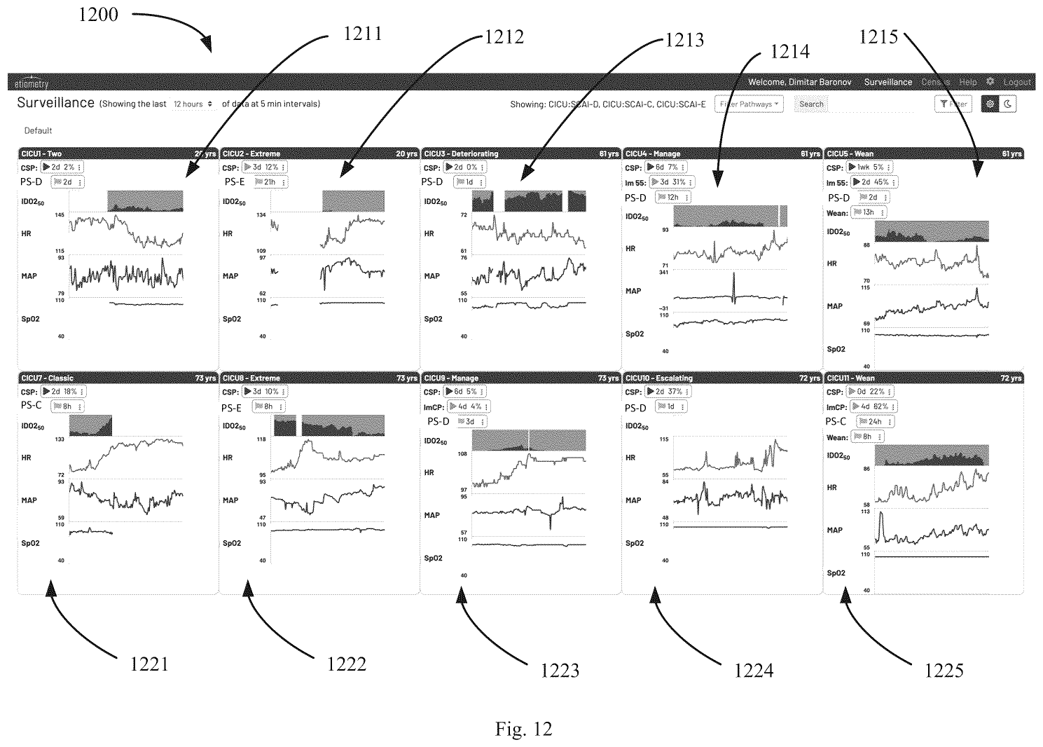

FIG. 12 schematically illustrates an embodiment of a graphical user interface 1200 displaying a plurality of individual graphical user interfaces (1211; 1212; 1213; 1214; 1215; 1221; 1222; 1223; 1224; 1225) in which each individual graphical user interface is for a corresponding individual patient. In illustrative embodiments, each individual graphical user interface shows data taken from its associated patient, which data defines at least in part a patient state for that patient. In some embodiments, each of the individual graphical user interfaces may be a graphical user interface as described in connection with FIG. 11, for example. Such a graphical user interface 1200 allows a medical professional (e.g., doctor or nurse) to quickly check-on and monitor a plurality of patients from one source, which would not be possible if each patient had a corresponding graphical user interface displayed, for example, on a corresponding plurality of computer screens, or that require the medical professional to navigate to each individual graphical user interface to show each such interface sequentially on a single computer screen.

Various embodiments may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P101. A system for event-driven patient monitoring and treatment of a patient experiencing cardiogenic shock, the system comprising:
a treatment device coupled to the patient and configured to administer a treatment to the patient under control of a computer;
a computer processor;
a memory coupled to the computer processor, the memory having computer-executable instructions stored thereon, which when executed by the computer processor, cause the computer processor to:
   receive patient-specific physiological data associated with the patient, the patient-specific data comprising at least a first physiological variable and a second physiological variable from the patient;
   define a state space with the first physiological variable on a first axis and the second physiological variable on a second axis, the state space partitioned into a plurality of possible clinical patient states of cardiogenic shock defined by a plurality of boundaries;
   determine, from the patient-specific physiological data and a treatment regimen for cardiogenic shock being administered to the patient, a current clinical patient state of the patient within the state space, the current clinical patient state being one of the plurality of possible patient states; and
   determine a selected treatment based on the current clinical patient state.
      P102: The system of P101, wherein the executable instructions, when executed by the computer processor, further cause the computer processor to, subsequent to and consequent to determining the selected treatment based on the current clinical patient state, send a signal to the treatment device coupled to the patient so that the signal causes the treatment device to administer the selected treatment to the patient, the treatment known to address the current clinical patient state.
      P103: The system of any of P101-P102, wherein the first physiological variable comprises lactate level and the second physiological variable comprises systolic blood pressure.
      P104: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a lactate level of zero.
   P105: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a lactate level of one.
   P106: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a lactate level of two.
   P107: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a lactate level of three.
   P108: The system of any of P101-P107, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a systolic blood pressure of 85.
   P109: The system of any of P101-P107, wherein the first physiological variable comprises lactate level and the second physiological variable comprises systolic blood pressure, and the current treatment regimen for cardiogenic shock comprises withholding medication and device treatment for cardiogenic shock, and:
the first patient state is defined in part as a systolic blood pressure of 90.
   P110: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and the second patient state comprises a first region in the state space and a second region in the state space, and the first region is defined in part as a lactate level having a lower boundary of zero.
   P111: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and the second patient state comprises a first region in the state space and a second region in the state space, and the first region is defined in part as a lactate level having a lower boundary of one.
   P112: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and the second patient state comprises a first region in the state space and a second region in the state space, and the first region is defined in part as a lactate level having a lower boundary of two.
   P113: The system of any of P101-P103, wherein the first physiological variable comprises lactate level and the second physiological variable comprises systolic blood pressure, and the current treatment regimen for cardiogenic shock comprises withholding medication and device treatment for cardiogenic shock, and the second patient state comprises a first region in the state space and a second region in the state space, and the first region is defined in part as a lactate level having a lower boundary of three.
   P114. The system of any of P110-P113, wherein the first physiological variable comprises lactate level and the second physiological variable comprises systolic blood pressure, and the current treatment regimen for cardiogenic shock comprises withholding medication and device treatment for cardiogenic shock, and the second patient state comprises a first region in the state space and a second region in the state space, and the first region is defined in part as a lactate level having an upper boundary of one.
   P201. A system for event-driven patient monitoring and treatment of a patient experiencing cardiogenic shock and subject to a current treatment regimen for cardiogenic shock, the system comprising:
a set of treatment devices coupled to the patient, each treatment device configured to administer a treatment to the patient under control of a computer;
a computer processor;
a memory coupled to the computer processor, the memory having computer-executable instructions stored thereon, which when executed by the computer processor, cause the computer processor to:
   receive patient-specific physiological data associated with the patient, the patient-specific data comprising at least a first physiological variable and a second physiological variable from the patient;
   define a state space with the first physiological variable on a first axis and the second physiological variable on a second axis, the state space partitioned into a plurality of possible clinical patient states of cardiogenic shock defined by a plurality of boundaries;
   determine, from the patient-specific physiological data and the current treatment regimen for cardiogenic shock being administered to the patient, a current clinical patient state of the patient within the state space, the current clinical patient state being one of the plurality of possible patient states;
   determine a set of current probabilities, each current probability of the set of current probabilities defining a probability of the patient transitioning from the current patient state to another patient state of the plurality of possible patient states under the current treatment regimen being applied to the patient;
   determine a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
   determine a selected treatment regimen from among the current treatment regimen and the set of alternative treatments, the selected treatment regimen being a treatment most likely, as determined by the set of alternative probabilities, to cause the patient to transition to a preferred patient state; and
   send a signal to the set of treatment devices coupled to the patient so that the signal causes the treatment devices to administer the selected treatment regimen to the patient, the treatment known to address the current clinical patient state.
      P202. The system of P201, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient state (D) is a preferred patient state relative to the fifth patient state, and
   the third patient state (C) is a preferred patient state relative to the fourth patient state and the fifth patient state, and
   the second patient state (B) is a preferred patient state relative to the third patient state and the fourth patient state and the fifth patient state, and
   the first patient state (A) is a preferred patient state relative to the second patient state and the third patient state and the fourth patient state and the fifth patient state.
      P203. The system of P201, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient (A) state is defined as lactate level between zero mmol/L and 2 mmol/L, with systolic blood pressure equal to or greater than 90 mmHg;
a second patient state (B) is defined as a first region having a lactate level between zero mmol/L and 2 mmol/L, and a systolic blood pressure between 60 mmHg and 90 mmHg, and a second region with a lactate level between 2 mmol/L and 5 mmol/L with systolic blood pressure equal to or greater than 90 mmHg;
a third patient state (C) is defined as lactate level between 2 mmol/L and 5 mmol/L, with systolic blood pressure between 60 mmHg and 90 mmHg;
a fourth patient state (D) is defined as lactate level between 5 mmol/L and 10 mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate level equal to or greater than 10 mmol/L with systolic blood pressure greater than zero mmHg;
in which:
   the fourth patient state (D) is a preferred patient state relative to the fifth patient state, and
   the third patient state (C) is a preferred patient state relative to the fourth patient state and the fifth patient state, and
   the second patient state (B) is a preferred patient state relative to the third patient state and the fourth patient state and the fifth patient state, and
   the first patient state (A) is a preferred patient state relative to the second patient state and the third patient state and the fourth patient state and the fifth patient state.
      P204. The system of P201, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
   the fourth patient (D) state is a preferred patient state relative to the fifth patient state (E), and
   the third patient state (C) is a preferred patient state relative to the fourth patient state and the fifth patient state.
      P205. The system of P201, wherein the current treatment regimen for cardiogenic shock consists of a single treatment selected from (i) a single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 2 mmol/L, with systolic blood pressure equal to or greater than 90 mmHg;
a fourth patient state (D) is defined as a fifth region having lactate level between zero mmol/L and 2 mmol/L, with systolic blood pressure between 60 mmHg and 90 mmHg, and a sixth region having lactate level greater between 2 mmol/L and 10 mmol/L, and systolic blood pressure equal to or greater than 60 mmHg;
and a fifth patient state (E) is defined as a third region with lactate level greater than zero mmol/L, with systolic blood pressure between zero mmHg and 60 mmHg, and a fourth region with lactate level equal to or greater than 10 mmol/L with systolic blood pressure greater than zero mmHg;
in which:
   the fourth patient (D) state is a preferred patient state relative to the fifth patient state (E), and
   the third patient state (C) is a preferred patient state relative to the fourth patient state and the fifth patient state.
      P206. The system of P201, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient (D) state is a preferred patient state relative to the fifth patient state (E).
      P207. The system of P201, wherein the current treatment regimen for cardiogenic shock consists of two treatments selected from (i) a single medication and a single device treatment, (ii) two medications and zero device treatments, and (iii) zero medications two device treatments, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate equal to or greater than 10 mmol/L with systolic blood pressure greater than zero mmHg;
in which:
   the fourth patient (D) state is a preferred patient state relative to the fifth patient state (E).
      P208. The system of P201, wherein the current treatment regimen for cardiogenic shock consists of three treatments selected from (i) a single medication and two device treatments, (ii) two medications and a single device treatment, and (iii) three medications and zero device treatments, and (iv) zero medications and three device treatments and:
the first physiological variable comprises lactate level, and the second physiological variable comprises systolic blood pressure, and there is no overlap between the patient states and no gap between patient states; and wherein
and a fifth patient state (E) is defined lactate level greater than zero mmol/L, with systolic blood pressure greater than zero mmHg;
in which:
   the fifth patient (E) state is a preferred patient state relative to death of the patient.
      P209. A computer-implemented method of for event-driven patient monitoring and treatment of a patient experiencing cardiogenic shock and subject to a current treatment regimen for cardiogenic shock using a system comprising a treatment device coupled to the patient and configured to administer a treatment to the patient under control of a computer having a computer processor, and a memory coupled to the computer processor, the method comprising:
receiving, by the computer, patient-specific physiological data associated with the patient, the patient-specific data comprising at least a first physiological variable and a second physiological variable from the patient;
defining, by the computer, a state space with the first physiological variable on a first axis and the second physiological variable on a second axis, the state space partitioned into a plurality of possible clinical patient states of cardiogenic shock defined by a plurality of boundaries;
determining, by the computer from the patient-specific physiological data and the current treatment regimen for cardiogenic shock being administered to the patient, a current clinical patient state of the patient within the state space, the current clinical patient state being one of the plurality of possible patient states;
determining, by the computer, a set of current probabilities, each current probability of the set of current probabilities defining a probability of the patient transitioning from the current patient state to another patient state of the plurality of possible patient states under the current treatment regimen being applied to the patient;
determining, by the computer, a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
determining, by the computer, a selected treatment regimen from among the current treatment regimen and the set of alternative treatments, the selected treatment regimen being a treatment most likely, as determined by the set of alternative probabilities, to cause the patient to transition to a preferred patient state; and
sending, from the computer, a signal to the treatment device coupled to the patient so that the signal causes the treatment device to administer the selected treatment to the patient, the treatment known to address the current clinical patient state.
   P210. The computer-implemented method of P209, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient state (D) is a preferred patient state relative to the fifth patient state, and
   the third patient state (C) is a preferred patient state relative to the fourth patient state and the fifth patient state, and
   the second patient state (B) is a preferred patient state relative to the third patient state and the fourth patient state and the fifth patient state, and
   the first patient state (A) is a preferred patient state relative to the second patient state and the third patient state and the fourth patient state and the fifth patient state.
      P211. The computer-implemented method of P209, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient (A) state is defined as lactate level between zero mmol/L and 2 mmol/L, with systolic blood pressure equal to or greater than 90 mmHg;
a second patient state (B) is defined as a first region having a lactate level between zero mmol/L and 2 mmol/L, and a systolic blood pressure between 60 mmHg and 90 mmHg, and a second region with a lactate level between 2 mmol/L and 5 mmol/L with systolic blood pressure equal to or greater than 90 mmHg;
a third patient state (C) is defined as lactate level between 2 mmol/L and 5 mmol/L, with systolic blood pressure between 60 mmHg and 90 mmHg;
a fourth patient state (D) is defined as lactate level between 5 mmol/L and 10 mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate level equal to or greater than 10 mmol/L with systolic blood pressure greater than zero mmHg;
in which:
   the fourth patient state (D) is a preferred patient state relative to the fifth patient state, and
   the third patient state (C) is a preferred patient state relative to the fourth patient state and the fifth patient state, and
   the second patient state (B) is a preferred patient state relative to the third patient state and the fourth patient state and the fifth patient state, and
   the first patient state (A) is a preferred patient state relative to the second patient state and the third patient state and the fourth patient state and the fifth patient state.
      P212. The computer-implemented method of P209, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
   the fourth patient (D) state is a preferred patient state relative to the fifth patient state (E), and
   the third patient state (C) is a preferred patient state relative to the fourth patient state and the fifth patient state.
      P213. The computer-implemented method of P209, wherein the current treatment regimen for cardiogenic shock consists of a single treatment selected from (i) a single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 2 mmol/L, with systolic blood pressure equal to or greater than 90 mmHg;
a fourth patient state (D) is defined as a fifth region having lactate level between zero mmol/L and 2 mmol/L, with systolic blood pressure between 60 mmHg and 90 mmHg, and a sixth region having lactate level greater between 2 mmol/L and 10 mmol/L, and systolic blood pressure equal to or greater than 60 mmHg;
and a fifth patient state (E) is defined as a third region with lactate level greater than zero mmol/L, with systolic blood pressure between zero mmHg and 60 mmHg, and a fourth region with lactate level equal to or greater than 10 mmol/L with systolic blood pressure greater than zero mmHg;
in which:
   the fourth patient (D) state is a preferred patient state relative to the fifth patient state (E), and
   the third patient state (C) is a preferred patient state relative to the fourth patient state and the fifth patient state.
      P214. The computer-implemented method of P209, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient (D) state is a preferred patient state relative to the fifth patient state (E).
      P215. The computer-implemented method of P209, wherein the current treatment regimen for cardiogenic shock consists of two treatments selected from (i) a single medication and a single device treatment, (ii) two medications and zero device treatments, and (iii) zero medications two device treatments, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate equal to or greater than 10 mmol/L with systolic blood pressure greater than zero mmHg;
in which:
   the fourth patient (D) state is a preferred patient state relative to the fifth patient state (E).
      P216. The computer-implemented method of P209, wherein the current treatment regimen for cardiogenic shock consists of three treatments selected from (i) a single medication and two device treatments, (ii) two medications and a single device treatment, and (iii) three medications and zero device treatments, and (iv) zero medications and three device treatments and:
the first physiological variable comprises lactate level, and the second physiological variable comprises systolic blood pressure, and there is no overlap between the patient states and no gap between patient states; and wherein
and a fifth patient state (E) is defined lactate level greater than zero mmol/L, with systolic blood pressure greater than zero mmHg;
in which:
   the fifth patient (E) state is a preferred patient state relative to death of the patient.
      P217. A non-transitory computer-readable medium having computer-executable instructions stored thereon, the instructions executable by a computer having a computer processor as part of a system comprising a set of treatment devices coupled to the patient, each treatment device configured to administer a treatment to the patient under control of the computer, and a memory coupled to the computer processor, the instructions comprising:
instructions to cause the computer to receive patient-specific physiological data associated with the patient, the patient-specific data comprising at least a first physiological variable and a second physiological variable from the patient;
instructions to cause the computer to define a state space with the first physiological variable on a first axis and the second physiological variable on a second axis, the state space partitioned into a plurality of possible clinical patient states of cardiogenic shock defined by a plurality of boundaries;
instructions to cause the computer to determine, from the patient-specific physiological data and the current treatment regimen for cardiogenic shock being administered to the patient, a current clinical patient state of the patient within the state space, the current clinical patient state being one of the plurality of possible patient states;
instructions to cause the computer to determine a set of current probabilities, each current probability of the set of current probabilities defining a probability of the patient transitioning from the current patient state to another patient state of the plurality of possible patient states under the current treatment regimen being applied to the patient;
instructions to cause the computer to determine a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
instructions to cause the computer to determine a selected treatment regimen from among the current treatment regimen and the set of alternative treatments, the selected treatment regimen being a treatment most likely, as determined by the set of alternative probabilities, to cause the patient to transition to a preferred patient state; and
instructions to cause the computer to send a signal to the set of treatment devices coupled to the patient so that the signal causes the treatment devices to administer the selected treatment regimen to the patient, the treatment known to address the current clinical patient state.
   P218. The non-transitory computer-readable medium of P217, wherein:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient state (D) is a preferred patient state relative to the fifth patient state, and
   the third patient state (C) is a preferred patient state relative to the fourth patient state and the fifth patient state, and
   the second patient state (B) is a preferred patient state relative to the third patient state and the fourth patient state and the fifth patient state, and
   the first patient state (A) is a preferred patient state relative to the second patient state and the third patient state and the fourth patient state and the fifth patient state.
      P219. The non-transitory computer-readable medium of P217, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
   the fourth patient (D) state is a preferred patient state relative to the fifth patient state (E), and
   the third patient state (C) is a preferred patient state relative to the fourth patient state and the fifth patient state.
      P220. The non-transitory computer-readable medium of P217, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
   the fourth patient (D) state is a preferred patient state relative to the fifth patient state (E).

Various embodiments of this disclosure may be implemented at least in part in any conventional computer programming language. For example, some embodiments may be implemented in a procedural programming language (e.g., “C”), or in an object-oriented programming language (e.g., “C++”), or in Python, R, Java, LISP or Prolog. Other embodiments of this disclosure may be implemented as preprogrammed hardware elements (e.g., application specific integrated circuits, FPGAs, and digital signal processors), or other related components.

In an alternative embodiment, the disclosed apparatus and methods may be implemented as a computer program product for use with a computer system. Such implementation may include a series of computer instructions fixed either on a tangible medium, such as a non-transitory computer readable medium (e.g., a diskette, CD-ROM, ROM, FLASH memory, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of this disclosure may be implemented as a combination of both software (e.g., a computer program product) and hardware. Still other embodiments of this disclosure are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads.

The embodiments of the invention described above are intended to be merely exemplary; numerous variations and modifications will be apparent to those skilled in the art. Such variations and modifications are intended to be within the scope of the present invention as defined by any of the appended claims.

While the foregoing includes the best mode and, where appropriate, other modes of performing the disclosure, the disclosure should not be limited to specific apparatus configurations or method steps disclosed in this description of the preferred embodiment. Those skilled in the art will also recognize that the disclosure has a broad range of applications, and that the embodiments admit of a wide range of modifications without departing from the inventive concepts.

What is claimed is:

1. A system for event-driven patient monitoring and treatment of a patient experiencing cardiogenic shock and subject to a current treatment regimen for cardiogenic shock, the system comprising:
a set of treatment devices coupled to the patient, each treatment device configured to administer a treatment to the patient under control of a computer;
a computer processor;
a memory coupled to the computer processor, the memory having computer-executable instructions stored thereon, which when executed by the computer processor, cause the computer processor to:
receive patient-specific physiological data associated with the patient, the patient-specific data comprising at least a first physiological variable and a second physiological variable from the patient;
define a state space with the first physiological variable on a first axis and the second physiological variable on a second axis, the state space partitioned into a plurality of possible clinical patient states of cardiogenic shock defined by a plurality of boundaries;
determine, from the patient-specific physiological data and the current treatment regimen for cardiogenic shock being administered to the patient, a current clinical patient state of the patient within the state space, the current clinical patient state being one of the plurality of possible patient states;
determine a set of current probabilities, each current probability of the set of current probabilities defining a probability of the patient transitioning from the current patient state to another patient state of the plurality of possible patient states under the current treatment regimen being applied to the patient;
determine a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
determine a selected treatment regimen from among the current treatment regimen and the set of alternative treatments, the selected treatment regimen being a treatment most likely, as determined by the set of alternative probabilities, to cause the patient to transition to a preferred patient state; and
send a signal to the set of treatment devices coupled to the patient so that the signal causes the treatment devices to administer the selected treatment regimen to the patient, the treatment known to address the current clinical patient state.

2. The system of claim 1, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient state (D) is a preferred patient state relative to the fifth patient state, and
the third patient state (C) is a preferred patient state relative to the fourth patient state and the fifth patient state, and
the second patient state (B) is a preferred patient state relative to the third patient state and the fourth patient state and the fifth patient state, and
the first patient state (A) is a preferred patient state relative to the second patient state and the third patient state and the fourth patient state and the fifth patient state.

3. The system of claim 1, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient (A) state is defined as lactate level between zero mmol/L and 2 mmol/L, with systolic blood pressure equal to or greater than 90 mmHg;
a second patient state (B) is defined as a first region having a lactate level between zero mmol/L and 2 mmol/L, and a systolic blood pressure between 60 mmHg and 90 mmHg, and a second region with a lactate level between 2 mmol/L and 5 mmol/L with systolic blood pressure equal to or greater than 90 mmHg;
a third patient state (C) is defined as lactate level between 2 mmol/L and 5 mmol/L, with systolic blood pressure between 60 mmHg and 90 mmHg;
a fourth patient state (D) is defined as lactate level between 5 mmol/L and 10 mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate level equal to or greater than 10 mmol/L with systolic blood pressure greater than zero mmHg;
in which:
the fourth patient state (D) is a preferred patient state relative to the fifth patient state, and
the third patient state (C) is a preferred patient state relative to the fourth patient state and the fifth patient state, and
the second patient state (B) is a preferred patient state relative to the third patient state and the fourth patient state and the fifth patient state, and
the first patient state (A) is a preferred patient state relative to the second patient state and the third patient state and the fourth patient state and the fifth patient state.

4. The system of claim 1, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
the fourth patient (D) state is a preferred patient state relative to the fifth patient state (E), and
the third patient state (C) is a preferred patient state relative to the fourth patient state and the fifth patient state.

5. The system of claim 1, wherein the current treatment regimen for cardiogenic shock consists of a single treatment selected from (i) a single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 2 mmol/L, with systolic blood pressure equal to or greater than 90 mmHg;
a fourth patient state (D) is defined as a fifth region having lactate level between zero mmol/L and 2 mmol/L, with systolic blood pressure between 60 mmHg and 90 mmHg, and a sixth region having lactate level greater between 2 mmol/L and 10 mmol/L, and systolic blood pressure equal to or greater than 60 mmHg;
and a fifth patient state (E) is defined as a third region with lactate level greater than zero mmol/L, with systolic blood pressure between zero mmHg and 60 mmHg, and a fourth region with lactate level equal to or greater than 10 mmol/L with systolic blood pressure greater than zero mmHg;
in which:
the fourth patient (D) state is a preferred patient state relative to the fifth patient state (E), and
the third patient state (C) is a preferred patient state relative to the fourth patient state and the fifth patient state.

6. The system of claim 1, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient (D) state is a preferred patient state relative to the fifth patient state (E).

7. The system of claim 1, wherein the current treatment regimen for cardiogenic shock consists of two treatments selected from (i) a single medication and a single device treatment, (ii) two medications and zero device treatments, and (iii) zero medications two device treatments, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate equal to or greater than 10 mmol/L with systolic blood pressure greater than zero mmHg;
in which:
the fourth patient (D) state is a preferred patient state relative to the fifth patient state (E).

8. The system of claim 1, wherein the current treatment regimen for cardiogenic shock consists of three treatments selected from (i) a single medication and two device treatments, (ii) two medications and a single device treatment, and (iii) three medications and zero device treatments, and (iv) zero medications and three device treatments and:
the first physiological variable comprises lactate level, and the second physiological variable comprises systolic blood pressure, and there is no overlap between the patient states and no gap between patient states; and wherein
and a fifth patient state (E) is defined lactate level greater than zero mmol/L, with systolic blood pressure greater than zero mmHg;
in which:
the fifth patient (E) state is a preferred patient state relative to death of the patient.

9. A computer-implemented method of for event-driven patient monitoring and treatment of a patient experiencing cardiogenic shock and subject to a current treatment regimen for cardiogenic shock using a system comprising a treatment device coupled to the patient and configured to administer a treatment to the patient under control of a computer having a computer processor, and a memory coupled to the computer processor, the method comprising:
receiving, by the computer, patient-specific physiological data associated with the patient, the patient-specific data comprising at least a first physiological variable and a second physiological variable from the patient;
defining, by the computer, a state space with the first physiological variable on a first axis and the second physiological variable on a second axis, the state space partitioned into a plurality of possible clinical patient states of cardiogenic shock defined by a plurality of boundaries;
determining, by the computer from the patient-specific physiological data and the current treatment regimen for cardiogenic shock being administered to the patient, a current clinical patient state of the patient within the state space, the current clinical patient state being one of the plurality of possible patient states;
determining, by the computer, a set of current probabilities, each current probability of the set of current probabilities defining a probability of the patient transitioning from the current patient state to another patient state of the plurality of possible patient states under the current treatment regimen being applied to the patient;
determining, by the computer, a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
determining, by the computer, a selected treatment regimen from among the current treatment regimen and the set of alternative treatments, the selected treatment regimen being a treatment most likely, as determined by the set of alternative probabilities, to cause the patient to transition to a preferred patient state; and
sending, from the computer, a signal to the treatment device coupled to the patient so that the signal causes the treatment device to administer the selected treatment to the patient, the treatment known to address the current clinical patient state.

10. The computer-implemented method of claim 9, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient state (D) is a preferred patient state relative to the fifth patient state, and
the third patient state (C) is a preferred patient state relative to the fourth patient state and the fifth patient state, and
the second patient state (B) is a preferred patient state relative to the third patient state and the fourth patient state and the fifth patient state, and
the first patient state (A) is a preferred patient state relative to the second patient state and the third patient state and the fourth patient state and the fifth patient state.

11. The computer-implemented method of claim 9, wherein the current treatment regimen for cardiogenic shock comprises withholding medication and device treatment for cardiogenic shock, and:
the first physiological variable comprises lactate level, and the second physiological variable comprises systolic blood pressure, and there is no overlap between the patient states and no gap between patient states; and wherein
a first patient (A) state is defined as lactate level between zero mmol/L and 2 mmol/L, with systolic blood pressure equal to or greater than 90 mmHg;
a second patient state (B) is defined as a first region having a lactate level between zero mmol/L and 2 mmol/L, and a systolic blood pressure between 60 mmHg and 90 mmHg, and a second region with a lactate level between 2 mmol/L and 5 mmol/L with systolic blood pressure equal to or greater than 90 mmHg;
a third patient state (C) is defined as lactate level between 2 mmol/L and 5 mmol/L, with systolic blood pressure between 60 mmHg and 90 mmHg;
a fourth patient state (D) is defined as lactate level between 5 mmol/L and 10 mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate level equal to or greater than 10 mmol/L with systolic blood pressure greater than zero mmHg;
in which:
the fourth patient state (D) is a preferred patient state relative to the fifth patient state, and
the third patient state (C) is a preferred patient state relative to the fourth patient state and the fifth patient state, and
the second patient state (B) is a preferred patient state relative to the third patient state and the fourth patient state and the fifth patient state, and
the first patient state (A) is a preferred patient state relative to the second patient state and the third patient state and the fourth patient state and the fifth patient state.

12. The computer-implemented method of claim 9, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
the fourth patient (D) state is a preferred patient state relative to the fifth patient state (E), and
the third patient state (C) is a preferred patient state relative to the fourth patient state and the fifth patient state.

13. The computer-implemented method of claim 9, wherein the current treatment regimen for cardiogenic shock consists of a single treatment selected from (i) a single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 2 mmol/L, with systolic blood pressure equal to or greater than 90 mmHg;
a fourth patient state (D) is defined as a fifth region having lactate level between zero mmol/L and 2 mmol/L, with systolic blood pressure between 60 mmHg and 90 mmHg, and a sixth region having lactate level greater between 2 mmol/L and 10 mmol/L, and systolic blood pressure equal to or greater than 60 mmHg;
and a fifth patient state (E) is defined as a third region with lactate level greater than zero mmol/L, with systolic blood pressure between zero mmHg and 60 mmHg, and a fourth region with lactate level equal to or greater than 10 mmol/L with systolic blood pressure greater than zero mmHg;
in which:
the fourth patient (D) state is a preferred patient state relative to the fifth patient state (E), and
the third patient state (C) is a preferred patient state relative to the fourth patient state and the fifth patient state.

14. The computer-implemented method of claim 9, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient (D) state is a preferred patient state relative to the fifth patient state (E).

15. The computer-implemented method of claim 9, wherein the current treatment regimen for cardiogenic shock consists of two treatments selected from (i) a single medication and a single device treatment, (ii) two medications and zero device treatments, and (iii) zero medications two device treatments, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 60 mmHg;
and a fifth patient state (E) is defined as a third region with lactate level between zero mmol/L and 10 mmol/L, with systolic blood pressure between zero mmHg and 60 mmHg, and a fourth region with lactate equal to or greater than 10 mmol/L with systolic blood pressure greater than zero mmHg;
in which:
the fourth patient (D) state is a preferred patient state relative to the fifth patient state (E).

16. The computer-implemented method of claim 9, wherein the current treatment regimen for cardiogenic shock consists of three treatments selected from (i) a single medication and two device treatments, (ii) two medications and a single device treatment, and (iii) three medications and zero device treatments, and (iv) zero medications and three device treatments and:
the first physiological variable comprises lactate level, and the second physiological variable comprises systolic blood pressure, and there is no overlap between the patient states and no gap between patient states; and wherein
and a fifth patient state (E) is defined lactate level greater than zero mmol/L, with systolic blood pressure greater than zero mmHg;
in which:
the fifth patient (E) state is a preferred patient state relative to death of the patient.

17. A non-transitory computer-readable medium having computer-executable instructions stored thereon, the instructions executable by a computer having a computer processor as part of a system comprising a set of treatment devices coupled to the patient, each treatment device configured to administer a treatment to the patient under control of the computer, and a memory coupled to the computer processor, the instructions comprising:
instructions to cause the computer to receive patient-specific physiological data associated with the patient, the patient-specific data comprising at least a first physiological variable and a second physiological variable from the patient;
instructions to cause the computer to define a state space with the first physiological variable on a first axis and the second physiological variable on a second axis, the state space partitioned into a plurality of possible clinical patient states of cardiogenic shock defined by a plurality of boundaries;
instructions to cause the computer to determine, from the patient-specific physiological data and the current treatment regimen for cardiogenic shock being administered to the patient, a current clinical patient state of the patient within the state space, the current clinical patient state being one of the plurality of possible patient states;
instructions to cause the computer to determine a set of current probabilities, each current probability of the set of current probabilities defining a probability of the patient transitioning from the current patient state to another patient state of the plurality of possible patient states under the current treatment regimen being applied to the patient;
instructions to cause the computer to determine a set of alternative probabilities, each alternative probability of the set of alternative probabilities defining a probability of the patient transitioning from the current patient state to another patient state under an alternative treatment selected from a set of alternative treatments, each alternative treatment of the set of alternative treatments being distinct from the current treatment;
instructions to cause the computer to determine a selected treatment regimen from among the current treatment regimen and the set of alternative treatments, the selected treatment regimen being a treatment most likely, as determined by the set of alternative probabilities, to cause the patient to transition to a preferred patient state; and
instructions to cause the computer to send a signal to the set of treatment devices coupled to the patient so that the signal causes the treatment devices to administer the selected treatment regimen to the patient, the treatment known to address the current clinical patient state.

18. The non-transitory computer-readable medium of claim 17, wherein:
the first physiological variable comprises lactate level, and the second physiological variable comprises systolic blood pressure, and there is no overlap between the patient states and no gap between patient states; and wherein
a first patient state (A) is defined as lactate level between zero mmol/L and 3 mmol/L, with systolic blood pressure equal to or greater than 85 mmHg;
a second patient (B) state is defined as a first region having a lactate level between zero mmol/L and 3 mmol/L, and a systolic blood pressure between 55 mmHg and 95 mmHg, and a second region with a lactate level between 1 mmol/L and 6 mmol/L with systolic blood pressure equal to or greater than 85 mmHg;
a third patient state (C) is defined as lactate level between 1 mmol/L and 6 mmol/L, with systolic blood pressure between 55 mmHg and 95 mmHg;
a fourth patient state (D) is defined as lactate level between 4 mmol/L and 11 mmol/L, with systolic blood pressure greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient state (D) is a preferred patient state relative to the fifth patient state, and
the third patient state (C) is a preferred patient state relative to the fourth patient state and the fifth patient state, and
the second patient state (B) is a preferred patient state relative to the third patient state and the fourth patient state and the fifth patient state, and
the first patient state (A) is a preferred patient state relative to the second patient state and the third patient state and the fourth patient state and the fifth patient state.

19. The non-transitory computer-readable medium of claim 17, wherein the current treatment regimen for cardiogenic shock consists of a single treatment selected from (i) single medication for cardiogenic shock or (ii) a single device treatment for cardiogenic shock, and:
the first physiological variable comprises lactate level, and the second physiological variable comprises systolic blood pressure, and there is no overlap between the patient states and no gap between patient states; and wherein
a third patient state (C) is defined as lactate level between zero mmol/L and 3 mmol/L, with systolic blood pressure between 85 mmHg and 95 mmHg;
a fourth patient state (D) is defined as a fifth region having lactate level between zero mmol/L and 1 mmol/L, with systolic blood pressure between 55 mmHg and 95 mmHg, and a sixth region having lactate level greater than 1 mmol/L and systolic blood pressure equal to or greater than 85 mmHg;
and a fifth patient state (E) is defined as a third region with lactate level greater than zero mmol/L, with systolic blood pressure between zero mmHg and 55 mmHg, and a fourth region with lactate level greater than 9 mmol/L with systolic blood pressure greater than zero mmHg;
in which:
the fourth patient (D) state is a preferred patient state relative to the fifth patient state (E), and
the third patient state (C) is a preferred patient state relative to the fourth patient state and the fifth patient state.

20. The non-transitory computer-readable medium of claim 17, wherein the current treatment regimen for cardiogenic shock consists of two treatments selected from (i) a single medication for cardiogenic shock and a single device treatment for cardiogenic shock, (ii) two medications for cardiogenic shock and zero device treatments for cardiogenic shock, and (iii) zero medications for cardiogenic shock and two device treatments for cardiogenic shock, and:
the first physiological variable comprises lactate level, and the second physiological variable comprises systolic blood pressure, and there is no overlap between the patient states and no gap between patient states; and wherein
a fourth patient state (D) is defined having lactate level greater than zero mmol/L, with systolic blood pressure equal to or greater than 55 mmHg;
and a fifth patient state (E) is defined as a third region with lactate level between zero mmol/L and 11 mmol/L, with systolic blood pressure between zero mmHg and 65 mmHg, and a fourth region with lactate level greater than 9 mmol/L with systolic blood pressure greater than zero mmHg;
in which:
the fourth patient (D) state is a preferred patient state relative to the fifth patient state (E).
